(19) United States
(12) Patent Application Publication
(10) Pub. No.: US 2026/0098252 A1
(43) Pub. Date: Apr. 9, 2026

(54) PURIFICATION OF CIRCULAR DNAS FROM CELLS USING NANOCLAYS

(71) Applicant: TEXAS STATE UNIVERSITY, San Marcos, TX (US)

(72) Inventors: Drew Scott Sowersby, San Marcos, TX (US); Lysle Kevin Lewis, San Marcos, TX (US)

(73) Assignee: TEXAS STATE UNIVERSITY, San Marcos, TX (US)

(21) Appl. No.: 19/349,595

(22) Filed: Oct. 3, 2025

Related U.S. Application Data
(60) Provisional application No. 63/703,015, filed on Oct. 3, 2024.

(51) Int. Cl. C12N 15/10; B01J 20/12; B01J 20/28; B01J 20/30

(52) U.S. Cl. CPC C12N 15/1006; B01J 20/12; B01J 20/28007; B01J 20/28009; B01J 20/3071

(57) ABSTRACT

Disclosed herein is a method of purifying circular DNA from a sample by exposure of the sample to a nanoclay that preferentially adsorbs multiple contaminants, including non-circular DNA, RNA, proteins, and endotoxins, while leaving circular DNA substantially unbound in solution. The elution-free method also includes a step of separating nanoclay-contaminant complexes from the enriched circular DNA fraction. Also disclosed herein are compositions and systems operable for purifying circular DNA from a sample. Also disclosed herein are methods of refining, formulating or modifying a nanoclay for use in purifying circular DNA from a sample.

### CROSS-REFERENCE TO RELATED APPLICATIONS

This application claims priority to U.S. Provisional Patent Application No. 63/703,015, filed on Oct. 3, 2024. The entirety of the aforementioned applications is incorporated herein by reference.

### BACKGROUND

There remains a need for simplified and effective methods to purify circular DNAs, such as plasmid DNAs. Circular DNAs are foundational to modern biotechnology, supporting applications in gene therapy, DNA vaccination, recombinant protein manufacturing, genome editing (e.g., CRISPR/Cas systems), cell engineering, and molecular diagnostics. Across research, clinical, and industrial contexts, circular DNAs are indispensable for gene cloning, protein expression, mutagenesis, and the creation of genetically modified organisms. Existing methods for purification of circular DNAs typically rely on chromatography or other elution-based systems, which are costly, complex, and difficult to scale. Accordingly, there remains a need for simplified and more scalable approaches for circular DNA purification. The embodiments of the present disclosure are directed toward addressing this need.

### SUMMARY

In some embodiments, the present disclosure pertains to methods for purifying circular DNA from a sample. The methods include exposing a sample to a nanoclay under conditions in which the nanoclay preferentially binds one or more non-circular DNA components relative to circular DNAs in the sample. In some embodiments, the methods further include separating the exposed sample from the nanoclay, thereby yielding a fraction enriched in purified circular DNA.

In additional embodiments, the present disclosure pertains to compositions useful for purifying circular DNA. Such compositions include a nanoclay preparation that selectively adsorbs non-circular DNA components while maintaining circular DNA in solution.

In further embodiments, the present disclosure pertains to systems operable for purifying circular DNA. The systems may include a vessel containing a nanoclay composition as described herein and, in some embodiments, one or more components operable to expose the sample to the nanoclay and subsequently separate the nanoclay-contaminant complexes from the sample.

In certain embodiments, the present disclosure pertains to methods of refining (e.g., modifying) a nanoclay for use in circular DNA purification. Such methods may include, without limitation: (a) washing raw nanoclay with a solution selected from the group consisting of basic solutions, acidic solutions, chelating agents, detergents, organic solvents, or combinations thereof; (b) dispersing the washed nanoclay; and (c) isolating the nanoclay (e.g., a fraction of the nanoclay). In some embodiments, the methods of the present disclosure also include a step of (d) characterizing the refined nanoclay (i.e., the resulting nanoclay preparation).

### DESCRIPTION OF THE DRAWINGS

FIG. 1 illustrates a process for purifying circular DNA (e.g., plasmid DNA) from cellular components through the utilization of nanoclays and in accordance with various embodiments of the present disclosure.

FIG. 2 provides a formulation workflow for the refinement of raw (unrefined) nanoclay powders, which are also called neat powders. In certain embodiments, raw nanoclay powder is dispersed in an aqueous dispersion medium, washed with a high pH mixture, and subjected to mixing and/or sonication to generate a nanoclay suspension. Following passive or active sedimentation, an rNP fraction is collected from the upper phase, while a sedimenting fraction may optionally be discarded as waste or repurposed. The collected rNPs are pooled into a pre-formulated batch and may be adjusted for one or more of pH, conductivity, or rNP concentration. The batch is optionally subjected to quality control, which in some embodiments includes assessment of colloidal scattering ratio (CSR), sterility, and/or mass concentration, to yield a standardized, application-ready formulated batch. The shading of boxes indicates relative particle density.

FIGS. 3A-3B show sedimentation-based differentiation between neat (raw or unprocessed) and refined nanoclay particle suspensions (rNPs). FIG. 3A shows sedimentation comparison of nanoclay suspensions subjected to different levels of mechanical and chemical processing. The initial tube (far left) shows the suspension prior to sedimentation. After 24 hours, sample (a) demonstrates dense pellet formation after simple mixing in water, (b) shows a more loose pellet formation after sonication in water, and (c) exhibits a stable quasi-colloidal suspension indicative of refined nanoclay particles (rNPs). In (c), extensive dispersion results in a more stable upper-phase suspension with dramatically reduced settling. FIG. 3B shows Z-average hydrodynamic diameter (+SD) for separate lots of Neat vs. rNP preparations. Neat sample lots A-α exhibit significantly higher mean particle sizes with broader distributions, while rNPs maintain consistent sub-500 nm sizes across all lots. Three measurements were conducted for each sample.

FIG. 4 shows X-ray diffraction patterns confirming compositional differences before and after refinement of nanoclay powders. Powder X-ray diffraction (XRD) profiles of a commercially available raw nanoclay powder (bottom), the refined nanoclay particles (rNPs, middle), and the sedimenting waste fraction (top) demonstrate selective enrichment of halloysite and removal of quartz and other impurities. Reference patterns for halloysite (PDF 00-060-1517, top vertical ticks) and quartz (PDF 00-003-0427, bottom vertical ticks) are annotated below. The rNP trace shows increased relative intensity of halloysite peaks and reduced quartz-associated signals, while quartz enrichment is observed in the sedimenting fraction. These results indicate that the refinement process preferentially retains halloysite nanotubes in suspension while depleting crystalline silica contaminants.

FIGS. 5A-5B show optical characterization of neat (unprocessed) vs. refined nanoclay particle suspensions (rNPs). FIG. 5A shows representative UV-visible light spectra showing wavelength-dependent light scattering of a raw powder (solid line) and corresponding rNPs (dashed line). The rNP batches exhibit enhanced scattering at lower wavelengths, consistent with improved dispersion and reduced aggregation. The double arrow lines highlight the differences in spectra between raw and rNPs. FIG. 5B shows colloidal scattering ratio (CSR) values, defined in this experiment as the ratio of OD₂₅₀/OD₇₀₀, for multiple lots of commercially available raw powders (A-F) and their corresponding rNP preparations (rA-rF). rNPs consistently display higher CSR values, indicative of more colloidally stable suspensions.

FIG. 6 illustrates plasmid DNA purification workflow using rNPs through integration of refined nanoclay particles (rNPs) into a preferred plasmid DNA purification pipeline. After cell lysis and crude lysate clarification, buffer compositions may be adjusted prior to addition of rNPs. Selective adsorption and induced flocculation cause aggregation of undesirable impurities such as genomic DNA (gDNA), RNA, protein, or LPS endotoxins. The clarified supernatant proceeds to filtration and final buffer exchange steps, yielding an enriched target plasmid DNA.

FIGS. 7A-7B show preferential removal of genomic DNA (gDNA) and RNA over plasmid DNA using rNPs. FIG. 7A is a UV absorbance assay comparing salmon gDNA depletion (removal) by neat nanoclay vs. rNPs. Samples treated with rNPs achieve near-complete removal of gDNA at much lower concentrations, whereas neat clay exhibits limited removal efficiency. This confirms that refinement (as measured by CSR values) enhances nucleic acid binding performance. FIG. 7B uses agarose gel electrophoresis to show the effect of increasing rNP concentration on a low-copy plasmid lysate. Supernatants from treated samples were loaded to visualize unbound material. Genomic DNA and RNA are progressively depleted with increasing rNP dose, while supercoiled, open circular, and multimer plasmid DNA isoforms remain in solution, demonstrating selective impurity adsorption by rNPs.

FIGS. 8A-8B illustrate selective depletion of non-plasmid impurities by refined nanoclay particles (rNPs). FIG. 8A is an agarose gel comparing untreated and rNP-treated RNase-free E. coli lysates, revealing complete removal of genomic DNA (gDNA) and RNA, while preserving plasmid DNA (pDNA) isoforms. The pDNA yield and band integrity remain largely unaffected following rNP treatment. ** Genomic DNA levels in final batches consistently measure below 1 pg/μL using a highly sensitive 16S rRNA gene PCR assay. FIG. 8B shows a colorimetric BCA assay demonstrating dose-dependent depletion of protein (removal of purple color) from both a solution of pure BSA and from a complex E. coli lysate upon rNP addition. Increasing clay concentration results in near-total protein removal, confirming broad protein-binding capacity of the refined nanoclay particles. BSA, bovine serum albumin protein control.

FIG. 9 illustrates time-dependent removal of RNA and genomic DNA from an RNase-free high copy pDNA lysate. The agarose gel demonstrates the effect of incubation time on rNP-mediated removal of RNA and genomic DNA from a high-copy plasmid DNA preparation. Samples were collected at the indicated timepoints (0-90 min) following treatment with rNPs. gDNA and RNA signals progressively diminish over time, while plasmid DNA isoforms (supercoiled, open circular, multimer) are retained in the supernatant. This time-course confirms the kinetic selectivity of rNPs for non-plasmid nucleic acids without impacting plasmid yield.

FIG. 10 shows selective recovery of circular 2μ plasmid DNA from S. cerevisiae extracts using rNPs. Shown is an agarose gel electrophoresis of an untreated (UN) and rNP-treated yeast cell extract. Lane C2 treatment was double the clay concentration of C1. Abundant high molecular weight gDNA, multiple isoforms of the endogenous 2μ plasmid DNA, and total degraded RNA are evident. Treatment with rNPs results in the depletion of gDNA and RNA while preserving the plasmid DNA isoforms in the supernatant. Increasing rNP concentration (C2) enhances removal of RNA with minimal loss of 2μ plasmid, indicating selective depletion of contaminants by rNPs in eukaryotic extracts.

FIG. 11 shows endpoint PCR analysis to assess genomic DNA contamination in plasmid preparations. PCR was performed using universal 16S rRNA primers to detect residual host E. coli genomic DNA (gDNA) in plasmid samples before and after treatment with rNPs. A serial dilution control series of purified E. coli gDNAs (1,000 pg/μL to 0.01 pg/μL) were included to establish detection sensitivity. Untreated samples from both methods showed strong gDNA signal across all dilutions. Treated samples showed complete loss of detectable gDNA amplification, even at the undiluted level.

### DETAILED DESCRIPTION OF THE INVENTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Plasmid DNA (pDNA) includes circular, typically supercoiled double-stranded DNA molecules ranging from a few thousand to several hundred thousand base pairs, distinct from chromosomal or genomic DNA (gDNA). pDNA serves as a cornerstone of modern biotechnology, with applications spanning gene cloning, expression studies, transgenic organism generation, recombinant protein production, gene therapy, DNA vaccination, genome editing (e.g., CRISPR/Cas systems), cell engineering, and molecular diagnostics. Because these applications often require highly pure material, demand for pDNA continues to rise sharply, particularly for clinical, medicinal, and GMP-grade preparations.

Conventionally, pDNA is produced by culturing plasmid-bearing cells (most commonly Escherichia coli cells) to high density, lysing the cells, and recovering the plasmid from the resulting lysate. This lysate is a complex mixture containing pDNA, gDNA, RNA species, proteins, lipopolysaccharides (LPS), and other cellular components. For pDNA intended for therapeutic or other regulated uses, these contaminants must be reduced to extremely low levels. Current purification methods typically employ three or more sequential unit operations—such as ion exchange chromatography, affinity chromatography, hydrophobic interaction chromatography, size exclusion chromatography, tangential flow filtration, and ultrafiltration—each targeting a subset of contaminants.

Despite their effectiveness, the current workflows for pDNA purification suffer from several critical limitations, such as high costs, complexity, yield loss, scalability constraints, and throughput limitations. In particular, typical pDNA purification workflows suffer from high costs because they usually require specialized chromatography instruments, resins and filtration modules that are expensive. Moreover, resin performance degrades with use.

Additionally, typical pDNA purification workflows are complex because they require multiple unit operations, extensive setup, cleaning, and validation. Existing pDNA purification workflows also suffer from yield loss because each purification step can result in substantial product loss, with conventional workflows sometimes discarding 40-70% of the starting pDNA.

Moreover, pDNA purification workflows have scalability constraints because chromatographic and filtration systems that function well at the laboratory scale can be difficult or uneconomical to scale to industrial batch sizes. Existing pDNA purification workflows also have throughput limitations because extended process times and large equipment footprints can slow manufacturing and reduce facility productivity.

Efforts to improve pDNA efficiency have focused on incremental modifications to existing chromatography techniques or the development of specialized affinity ligands for pDNA capture. However, these solutions still depend on large-scale chromatography infrastructure and have not eliminated the need for multiple downstream operations.

There remains a strong need for more streamlined, scalable, and cost-effective methods of purifying circular DNAs (e.g., pDNAs) from various sources (e.g., bacterial and other cellular sources). Ideally, such a method would selectively remove non-circular DNA contaminants while preserving circular DNA (e.g., pDNA) integrity and yield, function without specialized chromatography equipment, minimize the number of processing steps, and be readily adaptable from small-scale research to industrial-scale manufacturing. Numerous embodiments of the present disclosure aim to address the aforementioned needs.

In some embodiments, the present disclosure provides methods for purifying circular DNA from a sample. In some embodiments, such methods include exposing the sample to a nanoclay such that the nanoclay preferentially binds to one or more non-circular DNA components of the sample relative to the circular DNAs in the sample. In some embodiments, the methods of the present disclosure also include a step of separating the exposed sample from the nanoclay, where the separated sample includes purified circular DNA. In more specific embodiments, the methods of the present disclosure include contacting the sample with a nanoclay under conditions that promote selective binding of non-circular DNA components (e.g., genomic DNA, chromosomal DNA, and/or linear fragments), while leaving circular DNA (e.g., plasmid DNA and/or extrachromosomal circular DNA) substantially unbound. Following this exposure step, the nanoclay and bound contaminants are removed by a suitable separation technique, yielding a clarified preparation enriched in purified circular DNA.

In some embodiments, the present disclosure pertains to methods of refining a nanoclay for use in purifying circular DNA from a sample. In some embodiments, such methods include: (a) washing raw nanoclay with a solution selected from the group consisting of basic solutions, acidic solutions, chelating agents, detergents, organic solvents, or combinations thereof; (b) dispersing the washed nanoclay; and (c) isolating the nanoclay. In some embodiments, the methods of the present disclosure also include a step of (d) characterizing the refined nanoclay. In more specific embodiments, the present disclosure pertains to methods of refining, formulating, or storing nanoclays to generate refined nanoclay particle (rNP) preparations with enhanced biomolecule adsorption properties. Such refinements may improve particle size distribution, surface chemistry, colloidal stability, or lot-to-lot reproducibility, thereby tailoring the nanoclays for use in purification processes. In some embodiments, these rNP batches are employed to selectively remove non-circular DNA components (e.g., genomic DNA, chromosomal DNA, and/or RNA) from biological samples derived from bacterial, yeast, mammalian, or other cells, while preserving circular DNA, such as plasmid DNA or extrachromosomal circular DNA. In some embodiments, separation of the nanoclay-non-circular DNA component (e.g., contaminant) complexes yields a preparation enriched in purified circular DNA. It is to be understood that the separated sample (e.g., a liquid phase sample) includes purified circular DNA.

In some embodiments, the present disclosure provides compositions and systems for purifying circular DNA from a sample. The compositions include a nanoclay operable to preferentially bind non-circular DNA components of the sample relative to the circular DNAs in the sample. Such compositions may leave circular DNA in the sample substantially unbound. In certain embodiments, the system further includes a vessel containing the nanoclay. In some embodiments, the nanoclay may be in dry or suspension form. In some embodiments, the vessel may also include mixing or agitation features, for direct use in purification workflows.

In some embodiments, the present disclosure provides methods of modifying nanoclays for use in circular DNA purification. Such methods may include, for example: (a) washing raw nanoclay with a solution selected from the group consisting of basic solutions, acidic solutions, chelating agents, detergents, organic solvents, or combinations thereof; (b) dispersing the washed nanoclay (e.g., dispersion into a fluid medium); and (c) isolating (e.g., classifying) the nanoclay to obtain a desired particle fraction. Additional modifications, such as acid washes, chelation, surface functionalization, or size-based separation, may also be employed to tailor the nanoclay for selective adsorption performance. In some embodiments, the methods of the present disclosure also include a step of (d) characterizing the refined nanoclay.

As described in greater detail below, the present disclosure encompasses methods, compositions, and systems with a wide range of embodiments. The scope of the invention is not limited to the particular examples provided, but instead extends to variations, modifications, and equivalents that achieve the same purpose of selectively purifying circular DNA from complex biological samples.

### Samples

The methods, compositions, and systems of the present disclosure can be applied to purify circular DNA from a wide variety of samples. In some embodiments, the sample is a biological sample. In certain embodiments, the biological sample includes a cellular extract. In some embodiments, the cellular extract includes, without limitation, bacterial cell extracts (e.g., Escherichia coli), yeast cell extracts, eukaryotic cell extracts, mammalian cell extracts, or combinations thereof. In other embodiments, the sample may include blood, plasma, serum, saliva, tissue homogenates, viral preparations, or environmental samples containing microbial DNA. In some embodiments, the sample is a chemically or mechanically lysed preparation, a clarified fermentation broth, or another partially processed mixture containing circular DNA.

As used herein, the terms “cell extract” and “lysate” are used interchangeably and refer to preparations obtained from cells by any method of disruption, including but not limited to chemical lysis, enzymatic digestion, or mechanical disruption (e.g., sonication, bead milling, and/or French press).

### Refinement, Conditioning, and Formulation of Nanoclays

In some embodiments, the present disclosure provides methods, compositions, and systems that utilize nanoclays for purifying circular DNA. As used herein, the term nanoclay refers to naturally occurring or processed aluminosilicate minerals with dimensions in the nanometer to sub-micrometer range.

The nanoclay component may include naturally occurring or synthetic aluminosilicate minerals, including but not limited to kaolinite group minerals, smectites, sepiolite, palygorskite, attapulgite, and synthetic analogs such as laponite. Morphologies may be tubular, plate-like, fibrous, or irregular. Nanoclays may be obtained from natural ores, mined deposits, or other geological sources, and need not be commercially available. Nanoclays may also be prepared from synthetic or laboratory-processed materials.

In certain embodiments, the nanoclay is refined to produce refined nanoclay particles (rNPs) with controlled particle size, surface chemistry, and colloidal stability. Refinement may include base washing, chelation, acid treatment, size classification, sonication, or combinations thereof, and can improve lot-to-lot reproducibility, adsorption efficiency, and selectivity for non-circular DNA components.

In some embodiments, the nanoclay includes halloysite nanotubes (HNTs). HNTs are naturally occurring aluminosilicate nanotubes with high surface-area-to-mass (or surface-area-to-volume) ratios and distinct lumenal and external surface chemistries that render them particularly effective for biomolecule adsorption. In other embodiments, the nanoclay includes montmorillonite, sepiolite, kaolinite, or mixtures thereof, optionally in combination with HNTs.

The nanoclay may be employed in several forms. In some embodiments, the nanoclay is provided as refined nanoclay particles (rNPs), fractionated to defined particle size distributions (e.g., approximately 1-500 nm in width and 100-1500 nm in length) and dispersed to form colloidally stable or quasi-colloidal suspensions. In other embodiments, the nanoclay is used as an unrefined (neat) or raw powder, optionally milled, sieved (e.g., through 200-400 mesh screens), or washed to remove coarse particulates prior to use. In some embodiments, the nanoclay is surface-modified, such as by silanation, polymer coating, ligand functionalization, or magnetic doping. In yet other embodiments, the nanoclay is supported or immobilized within a secondary structure, such as a membrane, porous monolith, or fibrous matrix.

In some embodiments, refinement of nanoclays involves one or more processes such as aqueous dispersion, high-shear mixing, sonication, mechanical disaggregation, differential sedimentation or classification, and removal of mineral impurities (e.g., quartz or feldspar) by adjustment of conductivity, pH, or other solution properties. Refinement may be carried out to improve one or more properties, including particle size distribution, purity, colloidal stability, handling characteristics, and lot-to-lot reproducibility.

In some embodiments, refinement is assessed using optical scattering metrics. For example, a colloidal scattering ratio (CSR) may be determined by measuring optical density at two non-absorbing wavelengths (e.g., 250 nm and 700 nm) at defined timepoints. CSR values within a preset range are indicative of quasi-colloidal behavior, reduced aggregation, and resistance to rapid sedimentation. In certain embodiments, CSR is used as a lot-qualification metric to identify rNP batches suitable for use in circular DNA purification.

### Exposure of Samples to Nanoclays

The exposure of a sample to a nanoclay may occur under a variety of conditions. For instance, in some embodiments, exposure includes mixing the sample and the nanoclay under conditions sufficient to promote contact between the nanoclay and the non-circular DNA components in the sample. In some embodiments, exposure is achieved by contacting the sample with the nanoclay and mixing under conditions sufficient to promote selective adsorption of non-circular DNA components and other aforementioned contaminants. Such exposure may include, without limitation, stirring, shaking, inverting, vortexing, sonication, or combinations thereof, and may be carried out manually or by automated systems in either batch or continuous formats.

In certain embodiments, exposure occurs at a temperature between about 4° C. and 40° C., under buffered aqueous conditions (e.g., Tris, phosphate, or HEPES buffers), and for a time ranging from a one minute to several hours, depending on the sample constitution and intended application. In some embodiments, the ratio of nanoclay to sample is adjusted to achieve preferential removal of non-circular DNA while maintaining high recovery of circular DNA.

The exposure of a sample to a nanoclay may be performed across a range of temperatures, depending on the sample and intended application. In some embodiments, exposure is conducted at approximately room temperature (e.g., about 18° C. to 25° C.). In certain embodiments, exposure is carried out at temperatures between about 4° C. and about 40° C., such as between about 15° C. and about 30° C., under conditions that preserve nanoclay structural integrity and maintain adsorption performance.

The amount of nanoclay employed during exposure may vary depending on the concentration of nucleic acids, the composition of the sample, and the desired extent of purification. In some embodiments, nanoclay is measured as a mass concentration and can range from about 0.1 mg/mL to about 200 mg/mL in the exposure mixture. In other embodiments, the mass-to-mass ratio of nanoclay to total nucleic acid ranges from about 1:1 to about 10,000:1, such as from about 10:1 to about 1,000:1.

In certain embodiments, the relative amount of nanoclay is expressed as a dose factor (DF), defined as the amount of nanoclay added relative to the estimated nucleic acid or plasmid DNA content of the sample. The dose factor may be determined empirically, calculated from cell density or yield estimates, or obtained using an integrated module for dose determination. The use of a DF enables reproducible tuning of selective adsorption across diverse sample types and processing scales and also accounts for the inherent variability of nanoclays derived from different geological sources or geographic domains.

In some embodiments, the dose factor is selected to preferentially remove non-circular DNA contaminants while maintaining recovery of circular DNA. In other embodiments, the dose factor is adjusted according to the sample origin (e.g., bacterial, yeast, or mammalian), the intended application (e.g., research-grade versus GMP-grade purification), or the downstream process (e.g., small-scale minipreps versus large-scale fermentations). In certain embodiments, the dose factor is scaled proportionally for use in either batch or continuous workflows, thereby enabling flexible application of the technology across laboratory, pilot, and industrial production settings.

Separation of Exposed Samples from Nanoclays

The separation of a sample from nanoclays may be performed using a variety of methods. In some embodiments, separation is achieved by centrifugation, filtration, gravity sedimentation, magnetic capture, or combinations thereof.

In certain embodiments, separation is carried out without elution of circular DNA from a solid support, such that the purified circular DNA remains in the supernatant while the nanoclay and bound contaminants are removed as a pellet, filter retentate, or captured fraction. For example, in some embodiments, centrifugation produces a settled nanoclay pellet while circular DNA remains in the clarified supernatant.

In other embodiments, separation is performed without centrifugation, such as by gravity sedimentation or filtration. For instance, gravitational settling may allow nanoclay aggregates to sediment over time, or filters may be used to retain nanoclay particulates while permitting circular DNA to pass through a membrane.

In some embodiments, the nanoclays are functionalized with magnetic materials to render them magnetically responsive. In such embodiments, separation may be accomplished by magnetic capture of the nanoclay, enabling recovery of circular DNA from the remaining liquid fraction.

Following separation of the nanoclay from the sample, the resulting preparation is enriched in circular DNA. In some embodiments, no additional steps are required, as the separation itself provides a purified fraction suitable for downstream use. In other embodiments, the separated sample may be subjected to further treatment to enhance purity, adjust buffer composition, or meet specifications for particular applications such as research-grade, clinical-grade, or GMP-grade production.

### Further Treatment of Separated Samples

In some embodiments, the methods of the present disclosure further include treating the separated sample to adjust purity, composition, or yield according to downstream requirements. Such treatments may include, without limitation, enzymatic digestion (e.g., RNase treatment to remove RNA contaminants), precipitation or concentration of circular DNA, adjustment of ionic strength or pH, buffer exchange, or combinations thereof. The specific treatment pathway may be selected based on the intended application, such as research-grade preparations, clinical-grade materials, or GMP-compliant manufacturing.

In some embodiments, the separated sample is subjected to an additional round of treatment to further enhance purity or adjust composition. Such treatment may include repeating exposure to nanoclays under the same or modified conditions to remove residual contaminants, or applying one or more additional processing steps such as precipitation, filtration, ultrafiltration, or buffer exchange. In certain embodiments, iterative nanoclay treatments are employed to achieve higher levels of selectivity and yield, particularly for applications requiring clinical- or GMP-grade circular DNA.

In some embodiments, the treatment of a sample includes precipitation of the circular DNA from the sample. In some embodiments, the precipitation results in recovery and concentration of circular DNA from the sample. In some embodiments, the precipitation includes isopropanol or ethanol precipitation with or without salt carriers. In some embodiments, the precipitation occurs by membrane-based techniques, such as ultrafiltration, diafiltration, or tangential flow filtration (TFF), or combinations thereof.

In some embodiments, the treatment of a sample includes adjustment of a sample composition and ionic strength. In some embodiments, the adjustment also includes pH optimization to meet target storage or application requirements. In some embodiments, sample treatment includes buffer exchange into physiological or low-salt buffers, removal of low-molecular-weight impurities, and passage through sterile filtration devices (e.g., 0.22 μm) to achieve sterility for clinical or GMP-grade use.

In some embodiments, the separated and treated sample is further processed to reduce or remove residual protein or endotoxin contaminants, or to improve suitability for therapeutic use. In other embodiments, the circular DNA is formulated into a defined buffer system (e.g., Tris, TE, or physiological saline) and stored under conditions that preserve structural integrity, such as frozen, lyophilized, or liquid formats. In certain embodiments, sterilization or bioburden control is achieved through aseptic processing, filtration, or irradiation methods, depending on compatibility with the formulation.

### Nanoclays Variants and Formulations

The methods, compositions, and systems of the present disclosure can utilize a wide variety of nanoclays. In some embodiments, the nanoclays include, without limitation, halloysite, montmorillonite, sepiolite, laponite, kaolin, kaolinite, silicates, aluminosilicates, metakaolin, dickite, nacrite, smectite clays, bentonite, beidellite, nontronite, hectorite, saponite, palygorskite, attapulgite, phyllosilicates, or combinations thereof. In some embodiments, the nanoclay includes aluminosilicate materials. In some embodiments, synthetic nanoclays, including synthetic aluminosilicates such as laponite, are used.

Nanoclays of the present disclosure may be provided in various physical forms, including, without limitation, particles, nanoparticles, tubes, nanotubes, plate-like clays, fibrous clays, powders, hydrated clays, lyophilized clays, magnetic clays, multilayered clays, functionalized clays, raw clays, refined clays, refined nanoclay particles (rNPs), liquid suspensions, or combinations thereof. In certain embodiments, nanoclays are prepared as colloidal or quasi-colloidal suspensions with improved stability and handling characteristics relative to unrefined materials.

In some embodiments, nanoclays are sterilized or provided in a low-bioburden state. Sterilization may be achieved through autoclaving, gamma irradiation, aseptic processing, or membrane filtration. In other embodiments, nanoclays are lyophilized for long-term storage and reconstituted prior to use. In certain embodiments, nanoclays are at least about 95% pure by weight.

In some embodiments, nanoclays are reusable after a washing or regeneration step. In other embodiments, nanoclays are immobilized in frameworks such as porous membranes, fibrous matrices, or monoliths to permit reuse in batch or continuous purification workflows.

In some embodiments, the nanoclays include refined nanoclay particles (rNPs). In some embodiments, the rNPs are refined by washing nanoclays with a solution that includes, without limitation, basic solutions, acidic solutions, chelating agents, detergents, organic solvents, or combinations thereof. In certain embodiments, nanoclays are refined to produce rNPs. Refinement may include, without limitation, washing with basic solutions (e.g., NaOH, KOH, NH₄OH), chelation, acid treatment, mechanical disaggregation, sonication, or differential sedimentation. In some embodiments, rNPs may exhibit particle diameters less than about 100 nm, widths from about 1 nm to about 500 nm, and lengths from about 100 nm to about 1,500 nm, with average particle lengths between about 10 nm and about 1,000 nm.

In some embodiments, nanoclays are functionalized to modify surface chemistry, hydrophilicity, or charge. Functionalization may include, without limitation, polymers, peptides, silanes, ligands, anionic or cationic polymers, magnetic nanoparticles, or combinations thereof. In certain embodiments, magnetic functionalization enables recovery by magnetic separation. In some embodiments, the nanoclay may be functionalized with cationic polymers, peptides, or ligands to enhance adsorption of non-circular DNA components.

In some embodiments, nanoclays are provided as suspensions in aqueous or buffered solutions, such as Tris, phosphate-buffered saline, HEPES, or combinations thereof. Nanoclays may also be combined with stabilizers, including polymers, cryoprotectants, or surfactants, to enhance storage and dispersion stability. In some embodiments, refined nanoclay suspensions are characterized by a colloidal scattering ratio (CSR) at least two-fold higher than the corresponding raw nanoclay (e.g., that of unrefined nanoclay suspensions), indicating improved dispersion and reduced sedimentation.

In some embodiments, refined nanoclay suspensions are characterized by a colloidal scattering ratio (CSR), defined as the ratio of optical density at a shorter non-absorbing wavelength (e.g., about 250-320 nm) to a longer non-absorbing wavelength (e.g., about 700 nm). In certain embodiments, measurement at 250/700 nm provides a robust index of dispersion stability. Neat or unrefined nanoclays typically exhibit CSR values of about 1 to about 2 under such conditions, whereas refined nanoclay particles (rNPs) yield CSR values substantially higher, often exceeding about 5. In some embodiments, rNPs are defined by CSR values at least two-fold, three-fold, or more compared to their unrefined counterparts, indicative of improved dispersion quality and reduced sedimentation.

The concentration of nanoclays in a sample may vary depending on application. In some embodiments, concentrations are in the range of about 0.1 mg/mL to about 200 mg/mL. In other embodiments, the dose is defined relative to nucleic acid content, such as at least about 1.0×10⁻⁵ grams of nanoclay per A₂₆₀ unit of nucleic acid present in the sample.

In certain embodiments, nanoclays are formulated for specific applications. Formulations may include concentrated suspensions obtained by evaporation, ultrafiltration, or tangential flow filtration (TFF), followed by buffer exchange into defined media. Formulations may also include ionic strength modifiers or stabilizing agents to preserve adsorption capacity and long-term functionality.

Nanoclays and rNP formulations of the present disclosure may be qualified by physical and functional metrics, including, without limitation: visual uniformity; particle size and dispersity measured by light scattering or microscopy; zeta potential in neutral buffers; CSR values relative to unrefined clays; sedimentation index measurements; conductivity and pH confirmation; sterility or bioburden testing; and adsorption assays confirming selective removal of non-circular DNA while retaining circular DNA in solution.

In some embodiments, the nanoclays of the present disclosure are functionalized with a magnetic material. In some embodiments, the magnetic materials include magnetic nanoparticles.

### Circular DNAs

The methods, compositions, and systems of the present disclosure can be utilized to purify a variety of circular DNAs from samples. In some embodiments, the circular DNAs include, without limitation, extrachromosomal circular DNAs (eccDNAs), plasmid DNA (pDNA), viral episomes, synthetic minicircle DNAs, or combinations thereof. In certain embodiments, the circular DNA includes plasmid DNA, including plasmids used for gene therapy, DNA vaccination, genome editing, recombinant protein production, or molecular biology research.

In some embodiments, circular DNAs range in size from about 100 base pairs up to about 500,000 base pairs, including sub-ranges such as 100-50,000 base pairs, 100-5,000 base pairs, 100-2,500 base pairs, 100-1,000 base pairs, or 100-500 base pairs. In certain embodiments, the circular DNA is present in structural forms such as supercoiled, open circular (nicked), multimeric, or combinations thereof.

In some embodiments, the purified circular DNA obtained using the methods of the present disclosure constitutes at least 50% of the total DNA present in the sample. In other embodiments, the purified circular DNA fraction exceeds 70%, 80%, 90%, or 95% of the total DNA content. In some embodiments, the amount of nanoclay is determined by a dose factor (DF) expressed as grams of nanoclay per A₂₆₀ unit of nucleic acid in the sample. In some embodiments, the DF is established through a dose-response screen using a reference sample. In some embodiments, the purified circular DNA exhibits an A 260/280 absorbance ratio of at least about 1.80 to 1.90, consistent with high-purity nucleic acid suitable for clinical- or GMP-grade applications.

In some embodiments, the circular DNA includes mixtures of species differing in size or topology, including supercoiled, nicked, relaxed covalently closed circular (RCCC), or multimeric forms. In some embodiments, the methods of the present disclosure are operable to purify one or more of such forms from the sample.

### Non-Circular DNA Components

The nanoclays of the present disclosure can preferentially adsorb and remove a variety of non-circular DNA components (i.e., components other than circular DNA, including contaminating components), thereby enriching the circular DNA fraction. In some embodiments, such non-circular DNA components include, without limitation, non-circular DNA species (e.g., genomic DNA, chromosomal DNA, linear DNA fragments, degraded nucleic acids), RNA species (e.g., messenger RNA, ribosomal RNA, transfer RNA, or small RNAs), proteins (e.g., host cell proteins), endotoxins such as lipopolysaccharide (LPS), polysaccharides, carbohydrates, lipids, metabolites, cellular debris, contaminating components, or combinations thereof.

### Variations

The methods, compositions, and systems of the present disclosure can be applied in a variety of formats. In some embodiments, circular DNA purification occurs without reliance on elution steps. In other embodiments, the purification is implemented in a continuous-flow process or in a batch process and is scalable from milliliter-scale laboratory preparations to multi-liter or industrial-scale fermentations.

In some embodiments, the circular DNA purification methods of the present disclosure are repeated multiple times. In some embodiments, the circular DNA purification methods of the present disclosure include: (a) the exposure of a sample to a nanoclay; (b) the separation of the exposed sample from the nanoclay; (c) the re-exposure of the separated sample to a nanoclay; and (d) the separation of the re-exposed sample from the nanoclay. In some embodiments, step (a) occurs under higher salt concentrations than step (c). In some embodiments, step (a) occurs under salt concentrations of at least 0.1 mM, and step (c) occurs under salt concentrations of less than 50 mM. In some embodiments, the salt includes sodium chloride (NaCl). In other embodiments, the salt includes potassium chloride (KCl), magnesium chloride (MgCl), or calcium chloride (CaCl), without limitation.

In some embodiments, the multi-step circular DNA purification methods of the present disclosure reduce consumption of nanoclays by at least 50% compared to a single-step buffered purification process. In some embodiments, step (a) consumes less than 0.5 mg/mL of nanoclays. In some embodiments, step (c) preferentially removes residual RNA and endotoxin while circular DNA remains unbound.

### Systems

Embodiments of the present disclosure pertain to systems operable for purifying circular DNA from a sample. In some embodiments, the systems of the present disclosure include a nanoclay of the present disclosure. In some embodiments, the systems of the present disclosure also include a vessel containing the nanoclay. As set forth in more detail herein, the systems of the present disclosure can have numerous embodiments.

The systems of the present disclosure can include various vessels. For instance, in some embodiments, the vessel is operable to provide an environment for the exposure of a sample to a nanoclay. In some embodiments, the vessel includes an agitator operable to mix the sample with nanoclay. In some embodiments, the agitator includes, without limitation, an impeller, a stirrer, a shaker, or combinations thereof.

In some embodiments, the vessel includes a single-use disposable container. In some embodiments, the vessel is configured for batch processing of the sample. In some embodiments, the vessel is configured for continuous-flow processing of the sample.

The nanoclays of the present disclosure may be contained in vessels in various manners. For instance, in some embodiments, the nanoclays of the present disclosure are in the form of a dispersed suspension in an aqueous buffer in the vessel. In some embodiments, the nanoclays of the present disclosure are in the form of a dry powder operable to be reconstituted in a buffer. The systems of the present disclosure may also include additional components. For instance, in some embodiments, the systems of the present disclosure also include an apparatus operable to separate a sample exposed to a nanoclay from the nanoclay and thereby purify the circular DNA. In some embodiments, the apparatus includes, without limitation, a centrifugation apparatus, a filtration apparatus, a depth filtration unit, a membrane filter, a tangential-flow filtration (TFF) unit, a magnetic separator, a settling chamber configured to allow gravitational sedimentation of the nanoclay, or combinations thereof.

In some embodiments, the systems of the present disclosure also include an automated control system operable for controlling the purification of circular DNA from a sample. In some embodiments, the automated control system is operable to dose a nanoclay, monitor circular DNA purity, trigger a separation of the sample from the nanoclay, or combinations thereof.

In some embodiments, the systems of the present disclosure may be in the form of a kit. In some embodiments, the kit further includes instructions for using the nanoclay to purify circular DNA from the sample. In some embodiments, the instructions specify that no chromatographic elution step is required to recover the purified circular DNA. In some embodiments, the kit further includes a cell lysis buffer and a neutralization buffer for preparing a sample prior to the exposure of a sample to a nanoclay.

### Nanoclay Refinement Methods

Further embodiments of the present disclosure pertain to methods of refining (e.g., modifying) a nanoclay for use in purifying circular DNA from a sample. In some embodiments, such methods include: (a) washing raw nanoclay with a solution selected from the group consisting of basic solutions, acidic solutions, chelating agents, detergents, organic solvents, or combinations thereof; (b) dispersing the washed nanoclay; and (c) isolating the nanoclay. In some embodiments, the methods of the present disclosure also include a step of (d) characterizing the refined nanoclay. As set forth in more detail herein, the nanoclay refinement methods of the present disclosure can include numerous embodiments.

In some embodiments, the nanoclay refinement methods of the present disclosure also include a step of milling raw nanoclay before a washing step to reduce its particle size prior to refinement. In some embodiments, the nanoclay refinement methods of the present disclosure also include a step of washing raw nanoclay with a chelating agent to remove metal ions prior to a washing step.

In some embodiments, the nanoclay refinement methods of the present disclosure also include a step of conditioning the nanoclay with one or more buffers or salts to tune adsorption specificity. In some embodiments, the nanoclays are characterized by UV-Vis spectroscopy, X-ray diffraction (XRD), dynamic light scattering (DLS), or scanning electron microscopy (SEM) to confirm particle size, dispersion, and purity.

In some embodiments, the nanoclay refinement methods of the present disclosure also include a step of sterilizing the nanoclay after the isolating step. In some embodiments, the sterilizing occurs by a method that includes, without limitation, autoclaving, irradiation, or combinations thereof.

In some embodiments, the nanoclay refinement methods of the present disclosure also include a step of drying the nanoclay after the isolating step. In some embodiments, the drying occurs by a method that includes, without limitation, evaporative drying, lyophilization, or combinations thereof.

The nanoclay refinement methods of the present disclosure can include various washing steps. For instance, in some embodiments, the washing step includes treating the raw nanoclay with an alkaline solution. In some embodiments, the alkaline solution includes a concentration of about 0.1M to about 1 M. In some embodiments, the washing step takes place from about 10 minutes to about 60 minutes. In some embodiments, the washing step takes place at temperatures ranging from about 15° C. to about 30° C.

In some embodiments, the washing step includes treating raw nanoclay with a basic solutions. The nanoclay refinement methods of the present disclosure can utilize various basic solutions. For instance, in some embodiments, the basic solution includes, without limitation, NaOH, KOH, NH₄OH, or combinations thereof.

The nanoclay refinement methods of the present disclosure can disperse washed nanoclays in various manners. For instance, in some embodiments, the dispersion includes, without limitation, sonication, high-shear mixing, or combinations thereof. In some embodiments, the dispersion includes, without limitation, aqueous dispersion, high-shear mixing, sonication, differential sedimentation, or removal of mineral impurities (e.g., quartz) by adjustment of conductivity, pH, or other solution properties.

The nanoclay refinement methods of the present disclosure can isolate fractions of nanoclays in various manners. For instance, in some embodiments, the isolation step includes controlled sedimentation or ultrafiltration to select a desired size range.

### Advantages and Applications

The methods, compositions and systems of the present disclosure offers several advantages over existing circular DNA purification methods, compositions and systems. For instance, in some embodiments, the methods, compositions and systems of the present disclosure offer process simplification, which is capable of removing multiple contaminant classes from circular DNA in a single step. In some embodiments, the methods, compositions and systems of the present disclosure offer high yield retention, which offer minimal loss of circular DNA (e.g., pDNA) compared to conventional workflows. In some embodiments, the methods, compositions and systems of the present disclosure offer cost efficiency, which reduces or eliminates the need for expensive chromatographic resins. In some embodiments, the methods, compositions and systems of the present disclosure offer scalability, which may be especially effective at volumes ranging from small-scale research circular DNA (e.g., pDNA) preps to industrial-scale manufacturing batches. In some embodiments, the methods, compositions and systems of the present disclosure offer versatility, which is applicable to pDNA intended for research, diagnostic, and GMP-grade therapeutic applications.

In some embodiments, the methods, compositions and systems of the present disclosure can be implemented without the use of chromatography columns or other specialized infrastructure. Instead, in some embodiments, the methods, compositions and systems of the present disclosure may rely instead on simple mixing and separation techniques, such as centrifugation, filtration, sedimentation, or magnetic separation.

The selective binding profile observed with nanoclays (e.g., rNPs) is unexpected in the methods, compositions and systems of the present disclosure. The reason is because both circular DNA (e.g., pDNA) and many impurities share overall negative charge characteristics and could be predicted to bind to nanoclays.

As such, the methods, compositions and systems of the present disclosure can have numerous embodiments and applications. For instance, in some embodiments, the methods, compositions and systems of the present disclosure can be utilized to purify circular DNA for various applications. In some embodiments, such applications include, without limitation, therapeutics production, recombinant protein production, research and diagnostics, DNA vaccine production, gene therapy, cell therapy, immunotherapy, gene editing, protein production, or combinations thereof.

In some embodiments, the methods, compositions and systems of the present disclosure may utilize refined nanoclay particles (rNPs) in the purification of plasmid DNA from a cell extract. In some embodiments, the methods, compositions and systems of the present disclosure may utilize refined nanoclay particles in the removal of genomic DNA, RNA, protein, or endotoxins from a biological preparation. In some embodiments, the biological preparation includes a vaccine, gene therapy vector, or nucleic acid therapeutic. In some embodiments, the methods, compositions and systems of the present disclosure may utilize refined nanoclay particles in the purification of extrachromosomal circular DNA (eccDNA) from a cell extract. In some embodiments, the extrachromosomal circular DNA is obtained from a mammalian cell sample.

In some embodiments, the methods, compositions and systems of the present disclosure may utilize refined nanoclay particles in the purification of RNA from a cell lysate. In some embodiments, the nanoclay particles selectively adsorb DNA and protein contaminants while leaving the RNA in solution. In some embodiments, the methods, compositions and systems of the present disclosure may utilize refined nanoclay particles in the purification of a protein from a solution or cell extract. In some embodiments, the nanoclay particles selectively adsorb nucleic acids and endotoxins while leaving the target protein substantially unbound.

In some embodiments, the compositions of the present disclosure may be in the form of a formulation of suspensions (e.g., rNP suspensions) that can be stored, transported, and used as ready-to-deploy purification reagents. The ability to rapidly remove non-plasmid contaminants in a single step has the potential to dramatically streamline the production of high-purity circular DNA (e.g., pDNA) for diverse applications.

In some embodiments, the methods, compositions and systems of the present disclosure provide elution-free, column-free purification of circular DNA (e.g., pDNA). In specific embodiments, the methods of the present disclosure occur in an elution-free manner. Under selected conditions, the compositions of the present disclosure (e.g., rNPs) may preferentially adsorb non-plasmid components from a pDNA-containing sample while maintaining pDNA in solution. The compositions (e.g., rNPs) with bound contaminants may then be removed from the sample by a separation step (e.g., centrifugation, filtration, gravity sedimentation, and/or magnetic capture) without requiring any chromatographic binding or desorption/elution of pDNA. In certain embodiments, the process is executed as a single contact-and-separate operation.

In some embodiments, the methods, compositions and systems of the present disclosure operate in a host-agnostic manner. For instance, in some embodiments, the methods, compositions and systems of the present disclosure may be applicable to bacterial (e.g., E. coli), yeast (e.g., S. cerevisiae 2 μm plasmid systems), and mammalian cells, including but not limited to the purification of extrachromosomal circular DNAs (eccDNAs) from mammalian cell extracts. In some embodiments, the methods, compositions and systems of the present disclosure demonstrate topology and size selectivity, preserving supercoiled pDNA and/or eccDNA while preferentially removing gDNA, RNA, protein, LPS, and other contaminants, and optionally enriching the supercoiled or closed-circular isoforms.

In some embodiments, the methods, compositions and systems of the present disclosure can be utilized to enrich a supercoiled fraction of pDNA relative to nicked open circular (NOC) or linear forms of the plasmid by selectively removing co-purifying contaminants and/or conditions that disproportionately affect non-supercoiled species. In some embodiments, contact time, rNP dose, and solution conditions are tuned to further bias removal of high-molecular-weight nucleic acids (e.g., gDNA) over circular pDNA.

In some embodiments, rNPs in the compositions of the present disclosure may include one or more nanoclays, including halloysite, montmorillonite, sepiolite, kaolin/kaolinite, laponite, or combinations thereof. In some embodiments, rNPs in the compositions of the present disclosure are fractionated to defined size ranges and/or dispersity to tune adsorption behavior.

In some embodiments, rNPs in the compositions of the present disclosure are surface-modified or hybridized (e.g., polymer association, silane treatment, and/or ligand association) to alter surface chemistry, hydrophilicity, or charge characteristics without reliance on a specific chemistry. In some embodiments, mixed rNP systems are used to combine complementary binding preferences within a single operation.

In some embodiments, rNPs in the compositions of the present disclosure are rendered magnetizable by association with, coating on, or doping with magnetic particles, enabling magnetic separation after contaminant adsorption. In some embodiments, rNPs are immobilized on or within porous media, membranes, fibrous mats, or monoliths to create flow-through contactors in which contaminants are captured during passage while pDNA remains in the permeate/filtrate. In some embodiments, the operation does not require chromatographic binding and elution of the pDNA product.

In some embodiments, the methods of the present disclosure are carried out in batch mode by mixing rNPs with the sample followed by separation. In some embodiments, the methods of the present disclosure are implemented as staged operations, including pre-treatment of crude lysate and/or post-treatment of partially purified materials. In some embodiments, the methods of the present disclosure are executed continuously, for example via inline mixing of rNPs with a flowing stream and continuous separation (e.g., continuous centrifugation, settler/decant, crossflow filtration, and/or magnetic capture) within closed, single-use systems.

In some embodiments, solution pH, ionic strength, and/or additives (e.g., buffering agents, salts, chelators, and/or polymers) are adjusted to enhance preferential adsorption of non-plasmid components while retaining pDNA in solution. In some embodiments, one or more of contact time, temperature, rNP concentration, and mixing energy are selected to achieve a targeted balance of contaminant removal and pDNA yield.

In some embodiments, the compositions and systems of the present disclosure are in the form of a kit. In some embodiments, the kit includes: (i) a premeasured quantity of rNPs in dry (e.g., lyophilized or spray-dried) or suspended form; (ii) optionally, one or more conditioning buffers or additives configured to tune adsorption; and (iii) instructions for use describing rNP addition, mixing, and separation to obtain a pDNA-enriched supernatant. In some embodiments, kits are provided in pre-dosed sachets or cartridges scaled to culture volumes and packaged in sterile, single-use containers suitable for research or GMP environments.

In some embodiments, rNP lots in the compositions of the present disclosure are qualified by dispersion, sedimentation, and/or optical criteria to ensure lot-to-lot performance. In some embodiments, the qualification may include, without limitation, one or more of: (i) dispersion/colloid stability metrics; (ii) sedimentation behavior over a defined interval; and (iii) optical measurements at non-absorbing wavelengths indicative of scattering, all without specifying particular numeric thresholds. In some embodiments, a functional test demonstrates preferential removal of non-plasmid components from a standard matrix under defined contact conditions.

In some embodiments, the methods of the present disclosure may be utilized to refine raw nanoclay to produce reactive and/or selective refined nanoparticles (rNPs) with enhanced colloidal, structural, and chemical properties suitable for biomolecular adsorption. In some embodiments, the methods of the present disclosure may expose a pDNA-containing sample to rNPs under conditions that promote the preferential adsorption of non-plasmid DNA components and other contaminants to the rNPs. In some embodiments, the methods of the present disclosure may separate the rNPs and their bound contaminants from the remainder of the sample, thereby yielding pDNA supernatants of increased purity.

### ADDITIONAL EMBODIMENTS

Reference will now be made to more specific embodiments of the present disclosure and experimental results that provide support for such embodiments. However, Applicant notes that the disclosure herein is for illustrative purposes only and is not intended to limit the scope of the claimed subject matter in any way.

### Example 1. Scalable, Elution-Free Purification of Plasmid DNA Using Nanoclays

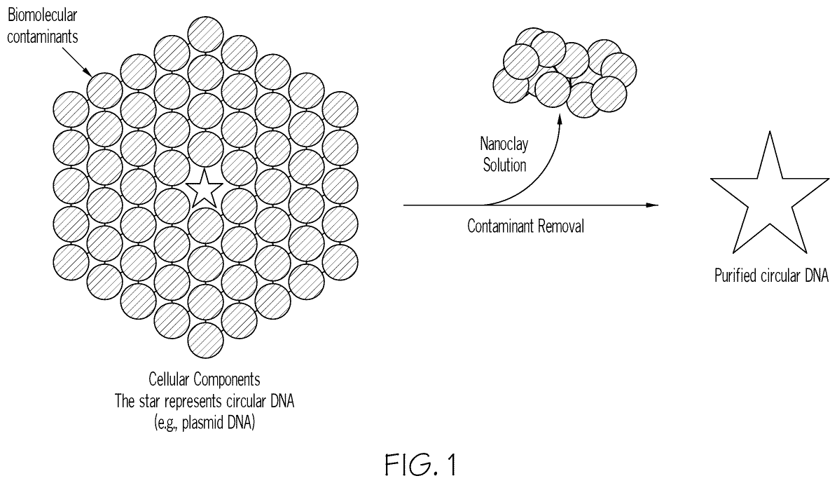

This Example relates to a process for using and refining raw nanoclay particles to create a colloidal fraction with enhanced structural and chemical properties, thereby allowing for efficient adsorption of unwanted non-plasmid DNA impurities. A schematic representation of the workflow is provided in FIG. 1, illustrating the general sequence of steps for exposure of cell lysate to nanoclay, separation of nanoclay-contaminant complexes, and recovery of purified circular DNA. This Example also relates to a method for employing the particles to purify circular plasmid DNAs and other circular DNA molecules or isoforms from cell lysates and extracts. The particles selectively bind unwanted cellular impurities, including genomic DNA, RNA and proteins, while permitting the recovery of intact circular plasmid DNA. Lysates may be derived from bacterial or eukaryotic cells and examples of each type of purification are described. This innovative approach enables efficient plasmid DNA isolation without reliance on conventional column chromatography equipment or elution steps.

More particularly, this Example pertains to the use of nanoclay particles—in either refined form, referred to herein as refined nanoclay particles (rNPs), or in certain embodiments as neat or raw powders—to selectively remove non-target contaminants from cellular extracts or lysates without the use of chromatographic elution.

Nanoclays, including but not limited to halloysite, kaolinite, kaolin, metakaolin, dickite, nacrite, montmorillonite, smectite clays, bentonite, beidellite, nontronite, hectorite, saponite, sepiolite, palygorskite, attapulgite, laponite, phyllosilicates, layered silicates, tubular aluminosilicates, hydrated aluminum silicates, fibrous silicate minerals, and naturally occurring or synthetic analogs thereof, possess unique physical and chemical characteristics at the nanoscale that make them highly effective for interacting with biological macromolecules.

When processed into rNPs, these particles can be optimized for properties such as narrow size distribution, high purity, and colloidal stability, which in turn can enhance binding selectivity, performance reproducibility, and scalability. However, refinement is not strictly required. In some cases, neat or unprocessed nanoclay powders are sufficient to achieve substantial contaminant removal, offering advantages in cost and simplicity.

The selective removal process described herein exploits the high surface-area-to-mass (or surface-area-to-volume) ratios, tunable surface chemistry, and differential binding characteristics of nanoclays to capture unwanted biological components, such as proteins, RNAs, chromosomal/genomic DNA, endotoxins, and other high-molecular-weight cellular materials, while leaving supercoiled circular DNA molecules, including plasmids and other extrachromosomal circular DNAs (eccDNAs), substantially unbound in the solution phase.

In certain embodiments, this Example provides methods for the selective purification of circular DNA molecules, including but not limited to plasmid DNA (pDNA) and extrachromosomal circular DNA (cccDNA), from complex biological mixtures. The methods utilize aluminosilicate nanoclay particles, preferably refined nanoclay particles (rNPs), which exhibit enhanced colloidal stability, reproducible particle size distributions, and selective adsorption properties. rNPs preferentially sequester non-target contaminants such as genomic DNA (gDNA), RNA, proteins, and endotoxins, while leaving target circular DNA substantially unbound in solution. The retained target DNA can then be recovered without chromatographic elution. In some embodiments, commercially available raw powders that inherently meet suitable performance thresholds may also be employed. However, rNPs represent the preferred materials of this Example.

### Example 1.1. Nanoclay Materials

The nanoclay component may include naturally occurring or synthetic aluminosilicate minerals, including but not limited to kaolinite group minerals, smectites, sepiolite, palygorskite, attapulgite, and synthetic analogs such as laponite. Morphologies may be tubular, plate-like, fibrous, or irregular, and may include halloysite nanotubes (HNTs) as a non-limiting example. In some embodiments, nanoclays may be obtained from natural ores, mined deposits, or other geological sources, and need not be commercially available. Additionally, the nanoclays may also be prepared from synthetic or laboratory-processed materials.

In various embodiments, the nanoclay may be used as refined nanoparticles (rNPs), which may be fractionated to defined particle size distributions (e.g., 1-500 nm width; 100-1500 nm length) and dispersed to form colloidally stable or quasi-colloidal suspensions. The nanoclays may also include unrefined, neat, or raw powders. Optionally, such powders may be milled, sieved, or washed to remove coarse particulates prior to use. The nanoclays may include surface-modified clays, which may be silanated, polymer-coated, ligand-functionalized, and/or magnetically doped. The nanoclays may also include supported clays, which may be immobilized within membranes, porous monoliths, and/or fibrous matrices.

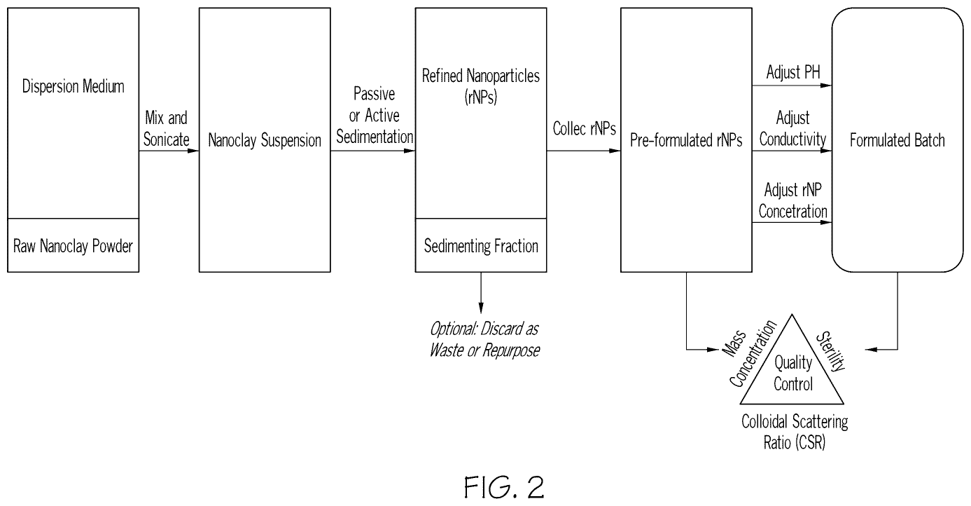

Nanoclays may be refined in various manners. For instance, refinement may involve one or more of aqueous dispersion, high-shear mixing, sonication, differential sedimentation, or removal of mineral impurities (e.g., quartz) by adjustment of conductivity, pH, or other solution properties. An illustrative schematic of representative refinement pathways is provided in FIG. 2, showing how raw nanoclay powders can be dispersed, disaggregated, and classified to yield refined nanoclay particles (rNPs) with improved colloidal stability and reproducibility.

### Example 1.2. Methods for Refining Nanoclays

Nanoclays may be refined to improve one or more of particle size distribution, purity, colloidal stability, handling characteristics, and lot-to-lot reproducibility. Refinement is preferrable in some embodiments, but is not strictly required. Neat or unrefined powders may be used directly in other embodiments. Where employed, refinement may include one or more of dispersion, washing, chemical conditioning, mechanical disaggregation, differential sedimentation or classification, removal of mineral impurities (e.g., quartz, feldspar), concentration, sterilization, and/or formulation.

### Example 1.3. Processing of Nanoclays

Nanoclays may be obtained from natural ores, mined deposits, or other geological sources, and need not be commercially available. Nanoclays may also be prepared from synthetic or laboratory-processed materials. Prior to refinement, powders may be screened or sieved (for example, through 200-400 mesh screens) to remove coarse particulates.

### Example 1.3.1. Aqueous Dispersion and Wetting

Aqueous dispersion and wetting of nanoclays may occur through the following steps:
1. Add powder to an aqueous dispersion medium (for example, water or buffered solution) at about 100-200 mg/mL.
2. Optional wetting aids or dispersion agents may be included at low levels (e.g., 0.001-0.1% v/v nonionic surfactant).
3. Mix by overhead stirrer, impeller, or rolling mixer for 1-120 minutes to uniformly wet the particles. This process can be extended where appropriate.

### Example 1.3.2. Chemical Conditioning and Washing

One or more conditioning steps may be used to disaggregate particles, remove accessory minerals, and tune or tailor surface charge. An operable temperature range for treatments is between 10° C. and 60° C., with a preferred range of about 20-40° C. to preserve nanoclay structural integrity and maintain adsorption characteristics.

### Example 1.3.2.1. Alkaline Wash

Add about 1-200 mM base (e.g, NaOH, KOH) to dispersion media for 1-120 minutes with mixing. Sediment nanoclay passively or via centrifugation and decant supernatant to rinse. Repeat as necessary.

### Example 1.3.2.2. Chelation Wash

Contact with about 1-100 mM chelator (e.g., EDTA, citrate) for 10-60 minutes to complex leachable metal ions. Sediment nanoclay passively or via centrifugation and decant supernatant to rinse. Repeat as necessary.

### Example 1.3.2.3. Salt Conditioning

Adjust ionic strength to 0.1-200 mM (e.g., NaCl, KCl, MgCl₂) to screen interparticle attractions during later disaggregation. Sediment nanoclay passively or via centrifugation and decant supernatant to rinse. Repeat as necessary.

### Example 1.3.2.4. Acid Wash

Add 1-200 mM acid (e.g, HCl, H₂SO₄) to dispersion media for 1-120 minutes with mixing. Sediment nanoclay passively or via centrifugation and decant supernatant to rinse. Repeat as necessary.

### Example 1.3.2.5. Mechanical Disaggregation

One or more mechanical energy inputs may be employed to dislodge and break apart aggregates of stacks, scrolls, or tactoids to aid in washing/rinsing or chemical conditioning processes. An operable temperature range treatments is about 10-60° C., with a preferred range of about 20-40° C. to preserve nanoclay structural integrity and maintain adsorption characteristics.

### Example 1.3.2.5 (a). High-Shear Mixing

Rotor-stator or high-speed impeller at about 3,000-20,000 rpm for 1-30 minutes.

### Example 1.3.2.5 (b) Sonication

Probe sonication at about 20-40 kHz, or 50-1500 W, continuously for short periods (e.g., under 3 minutes) or using pulsed duty cycle (e.g., 2-10 s on/5-30 s off) for 3-60 minutes with active cooling to maintain acceptable temperature as noted above.

### Example 1.3.2.5 (c) Mechanical Size Reduction

Mechanical size reduction may be performed using gentle bead milling with submillimeter media for short intervals (e.g., 1-10 minutes) to avoid excessive comminution, or by ball milling followed by sieving to obtain a desired particle size fraction. Such milling steps may be used as a stand-alone dispersion aid or as a preparatory step prior to chemical conditioning, washing, or other refinement processes.

### Example 1.3.2.5 (d). Differential Sedimentation and Classification

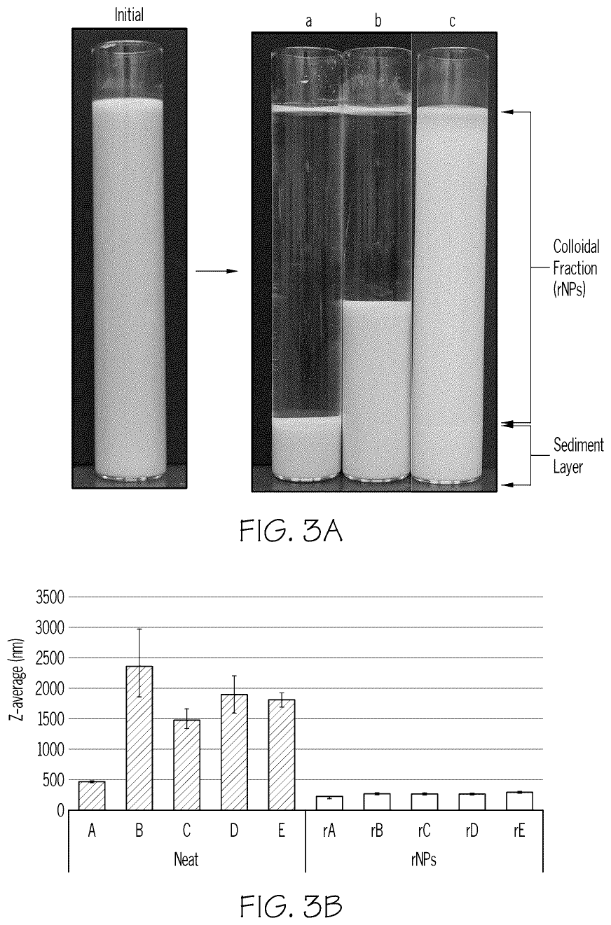

Refined fractions, typically corresponding to the upper-phase suspension enriched in finer or more colloidal particles, may be separated from columns, bottles, or tubes based on sedimentation behavior. Separation may be achieved passively under atmospheric gravity or accelerated using centrifugation. The process can be repeated or staged to progressively remove coarser aggregates and enrich the desired nanoclay fraction. As illustrated in FIGS. 3A-3B, refinement produces measurable differences in sedimentation behavior and particle size distribution. FIG. 3A shows visual sedimentation over 24 hours for suspensions of neat (raw) nanoclay, sonicated nanoclay, and refined nanoclay particles (rNPs). Neat suspensions rapidly form dense pellets, while rNPs exhibit stable quasi-colloidal suspensions with dramatically reduced settling. FIG. 3B presents hydrodynamic diameters (Z-average±SD) for multiple independent lots of neat versus rNP preparations. Neat lots (A-E) exhibit larger and more variable particle sizes, whereas rNPs consistently maintain sub-500 nm distributions, confirming refinement improves reproducibility and colloidal stability.

### Example 1.3.2.5 (d) (1). Gravity Classification

Allow dispersion to stand undisturbed for a defined window in tall vessels. With some nanoclays, this could take a few minutes or hours, while with others this process can take days or even weeks. Collect the upper “refined” zone that remains suspended beyond a set time, optionally discarding or repurposing the faster-settling fraction.

### Example 1.3.2.5 (d) (2). Centrifugation

Spin at about 50-1,000×g for about 5-60 minutes. Collect supernatant as the refined fraction while pelleting larger aggregates. Iterative passes may be used to tighten the size distribution.

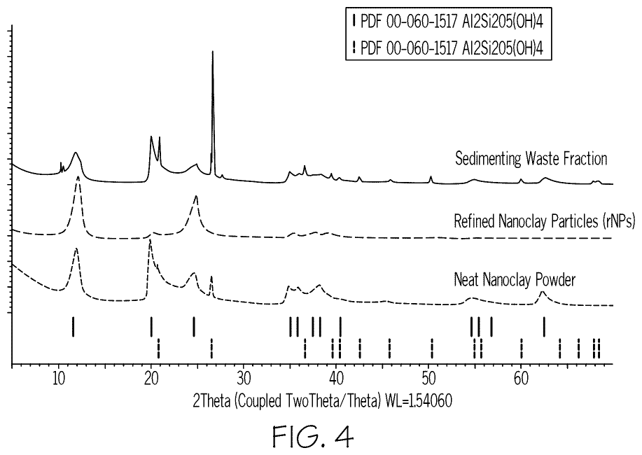

### Example 1.3.2.6. Removal of Accessory and Impurity Minerals

Where present, impurity (for example, quartz, feldspathic particles, iron oxides) may be reduced by a combination of pH conditioning, sedimentation, magnetic capture, or density separation. As a non-limiting example, FIG. 4 illustrates selective removal of quartz and accessory minerals from halloysite preparations. Adjustment of pH and sedimentation behavior enables enrichment of aluminosilicate nanotubes while reducing silica and other mineral impurities, thereby improving reproducibility and adsorption selectivity of the refined nanoclay particles (rNPs).

### Example 1.3.2.6 (a). PH-Mediated Selectivity

Alkaline dispersion as described (for example, 50-150 mM base) can differentially affect dispersion of aluminosilicate particles relative to dense silica grains, improving classification.

### Example 1.3.2.6 (b). Magnetic Capture (Optional)

If trace ferromagnetic particles are present, pass the dispersion through a magnetic separator and discard the captured fraction.

### Example 1.3.2.6 (c). Settling/Density Steps

When a nanoclay suspension is allowed to remain undisturbed for a defined period, the densest and largest particles will settle toward the bottom under the influence of gravity, while finer or less dense particles remain suspended in the upper phase. By carefully decanting and retaining this upper portion, it is possible to selectively enrich the sample in smaller or more colloidally stable particles. This step can be repeated, varied in duration, or combined with density modifiers to further refine particle size distribution before downstream use.

### Example 1.3.2.7. Concentration and Buffer Exchange

Refined fractions may be concentrated or exchanged into a formulation buffer.

### Example 1.3.2.7 (a). Concentration

Tangential flow filtration (e.g., 100-500 kDa MWCO), dead-end filtration, or gentle rotary evaporation or open air evaporation below about 40° C. may be utilized. rNPs may also be concentrated via high gravity centrifugation followed by resuspension in proper medium via mechanical stirring and/or sonication.

### Example 1.3.2.7 (b) Buffer Exchange

Nanoclay suspensions may be exchanged into water or a selected buffer (e.g., 1-200 mM Tris, HEPES, or other suitable buffering agents) and adjusted to a pH of about 6.0-9.0. This can be accomplished by repeated centrifugation and resuspension, tangential flow filtration, dialysis, or other diafiltration methods. Conductivity of the final suspension may be adjusted to about 0-50 mS/m (0-0.5 mS/cm) by altering ionic strength, depending on the desired dispersion stability, surface charge profile, and intended downstream interaction with biomolecules. Buffer composition, pH, and conductivity may be tailored to favor selective binding or release of contaminants while maintaining pDNA integrity.

### Example 1.3.2.7 (c). Sterilization and Bioburden Control

Depending on intended use, rNP suspensions or raw powder suspensions may be sterilized or provided as low-bioburden materials.

### Example 1.3.2.7 (c) (1) Sterile Filtration

Pass through of buffers and other liquids through 0.22-0.45 μm membrane filters.

### Example 1.3.2.7 (c) (2) Aseptic Processing

Prepare and package under aseptic conditions in sterile containers.

### Example 1.3.2.7 (c) (3) Alternative Methods

Gamma irradiation or autoclaving may be used where compatible with dispersion stability.

### Example 1.4. Nanoclay Formulations

Nanoclay formulations, such as rNP suspensions, may include: (a) buffer components to maintain pH about 6.0-9.0; (b) ionic strength modifiers (for example, 1-200 mM monovalent salts) to tune dispersion and downstream selectivity or elevate adsorption strength; and (c) optional stabilizers at low levels (for example, 0.001-0.05% nonionic agents) where consistent with downstream use. Final concentrations may range from about 1.0 mg/mL to over 100 mg/mL depending on the application and specific nanoclay.

### Example 1.4.1. Quality Control and Lot Qualification

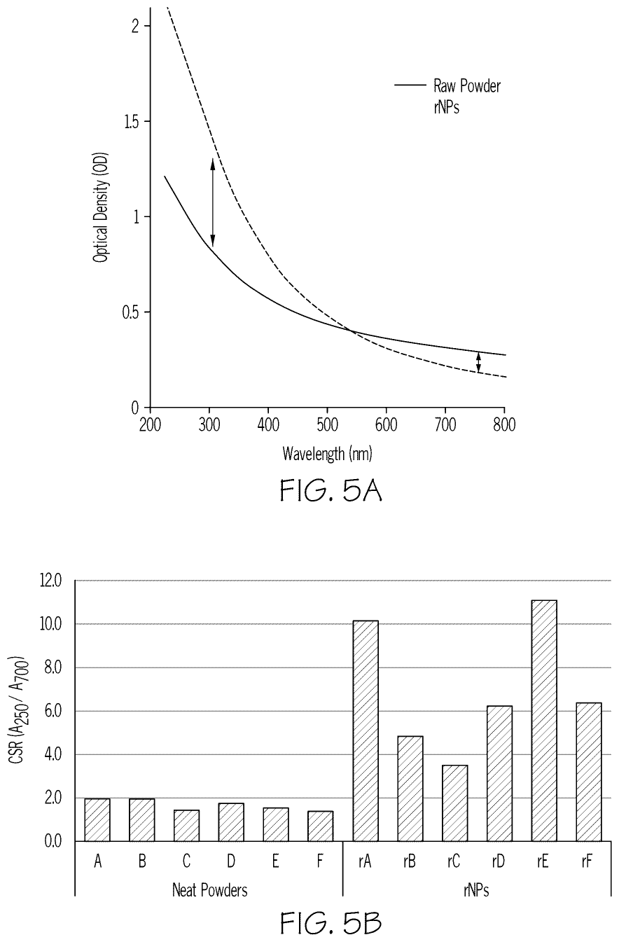

Lots may be qualified using physical and functional metrics. Such metrics may include, without limitation: (a) visual and color check (e.g., uniform white to off-white or beige appearance) and/or absence of visible aggregates after gentle mixing; (b) particle size and dispersity, such as dynamic light scattering (for example, Z-average and PDI) and/or microscopy (SEM, TEM, AFM) to verify size regime and morphology; (c) zeta potential, which is typically negative external potentials in neutral buffers, where values outside set bounds may indicate instability; (d) optical scattering metrics that determine a colloidal scattering ratio (CSR) by measuring optical density at two non-absorbing wavelengths (for example, about 250 nm and about 700 nm) at defined timepoints, where CSR within preset ranges indicates quasi-colloidal behavior and resistance to rapid sedimentation (refer to FIG. 5A for an example of the differences in optical density at different wavelengths of raw versus refined nanoclays and FIG. 5B for extreme differences in CSR values); (e) sedimentation index, which tracks absorbance or transmitted light at a fixed height over a defined interval (for example, 30-180 minutes) to quantify settling kinetics.

Alternatively, quality control can be done qualitatively through visual inspection, such as (f) conductivity and pH to confirm formulation setpoints; (g) sterility or bioburden testing, per intended use; and (h) functional binding testing in a standard matrix containing nucleic acids and proteins, confirming preferential removal of non-plasmid components under defined contact conditions while retaining circular DNA in solution.

### Example 1.5. Refinement of Nanoclays

In certain embodiments, separation operations described herein (e.g., centrifugation, filtration, sedimentation, and/or magnetic capture) are interchangeable and may be substituted with functionally equivalent technologies without departing from the scope of the present disclosure. Selection may be based on scale, equipment availability, desired cut size, or downstream requirements, provided that batch qualification and functional performance criteria are met.

An example of a nanoclay refinement workflow (illustrative, non-limiting) includes:
1. Disperse nanoclay powder at 50-200 mg/mL in ˜10 mM Tris pH 9.0 and mix for 5 minutes. Total volume is adjustable depending on batch size and sonication probe requirements.
2. Apply probe sonication for 1-5 minutes using a tip appropriate to vessel geometry, maintaining an energy density similar to that achieved at 60% amplitude (450 W for a 200 mL dispersion with a 13 mm probe). Probe size and time may be scaled proportionally with volume to achieve similar energy input per unit volume.
3. Repeat dispersion and sonication steps on additional samples to achieve desired total batch size and then transfer all suspensions to a single vessel.
4. Let suspension rest at RT for 12-36 hours and then transfer rNP-rich upper phase to an appropriate centrifuge bottle. Discard sediment phase.
5. Pellet the rNPs at 20 k×g for ˜15 minutes and resuspend the resulting pellet via mechanical stirring and/or sonication in appropriate buffer (e.g., 10 mM Tris, pH 8.0). The volume of buffer should be chosen based on formulated rNP concentration requirements. Typically, pellets are resuspended in 25-50% of the initial suspension volumes (Step 1).
6. Optional: autoclave batch at 121° C. for 15 minutes.
7. Acquire CSR, ascertain rNP concentration, and perform functional QC adsorption assay.

Another example of a nanoclay refinement workflow (illustrative, non-limiting) includes:
1. Disperse nanoclay powder at 50-200 mg/mL in ˜10 mM Tris pH 9 and mix for 5 minutes. Total volume is adjustable depending on batch size and sonication probe requirements.
2. Apply probe sonication for 1-5 minutes using a tip appropriate to vessel geometry, maintaining an energy density similar to that achieved at 60% amplitude (450 W for a 200 mL dispersion with a 13 mm probe). Probe size and time may be scaled proportionally with volume to achieve similar energy input per unit volume.
3. Repeat dispersion and sonication steps on additional samples to achieve proper batch size and then transfer all suspensions to a single vessel.
4. Transfer dispersions to centrifuge bottles and spin at 500×g for ˜15 minutes; retain supernatant as rNP fraction.
5. Pellet the rNPs at 20 k×g for ˜15 minutes and resuspend the resulting pellet via mechanical stirring and/or sonication in appropriate buffer (e.g., 10 mM Tris, pH 8.0). The volume of buffer should be chosen based on formulated rNP concentration requirements. Typically, pellets are resuspended in 25-50% of the initial suspension volumes (Step 1).
6. Optional: autoclave batch at 121° C. for 15 minutes.
7. Acquire CSR, ascertain rNP concentration, and perform functional QC adsorption assay.

Another example of a nanoclay refinement workflow (illustrative, non-limiting) with a wash/conditioning step (illustrative, non-limiting) includes:
1. Disperse nanoclay powder at 50-200 mg/mL in water and mix for 5 minutes. Total volume is adjustable depending on batch size and sonication probe requirements.
2. Add NaOH to ˜100 mM; mix thoroughly for ˜30 minutes at 15-30° C. and then pellet the nanoclay at 20 k×g for 15 minutes. Decant supernatant and resuspend pellet in same volume of water originally used. Repeat the process as necessary or until supernatant is clear or solubilized impurities are removed.
3. Add EDTA to ˜50 mM in roughly the same volume as decanted supernatant above to the clay pellet; mix thoroughly for 5 minutes at 15-30° C. and then pellet the nanoclay at 20 k×g for 15 minutes. Discard supernatant.
4. Add 10 mM NaOH to the pellet at the same supernatant volume removed in previous step. Dislodge clay from vessel walls and probe sonicate at 60% on a 450 W sonicator.
5. Spin at 500×g for 15 minutes; retain supernatant as refined nanoparticle (rNP) fraction.
6. Pellet the refined fraction at 20 k×g for 15 minutes and resuspend via sonication in appropriate buffer to maintain the pH of rNPs.
7. Optional: autoclave batch at 121° C. for 15 minutes.
8. Acquire CSR, ascertain rNP concentration, and perform functional QC adsorption assay.

Another example of a nanoclay refinement workflow (illustrative, non-limiting) with a wash/conditioning step (illustrative, non-limiting) includes:
1. Disperse nanoclay powder at 200 mg/mL in water and mix for 5 minutes. Total volume can be adjusted depending on batch size and sonication probe requirements.
2. Add EDTA to ˜100 mM; mix thoroughly for ˜5 minutes at 15-30° C. and then pellet the nanoclay at 20 k×g for 15 minutes. Measure volume and discard supernatant.
3. Add a measured volume of ˜100 mM NaOH to the pellet; mix thoroughly for ˜30 minutes at 15-30° C. and then pellet the nanoclay at 20 k×g for 15 minutes. Discard supernatant.
4. Add 10 mM NaOH to the pellet at the same supernatant volume removed in previous step. Dislodge clay from vessel walls and probe sonicate at 60% on a 450 W sonicator.
5. Spin at 500×g for 15 minutes; retain supernatant as refined nanoparticle (rNP) fraction.
6. Pellet the refined fraction at 20 k×g for 15 minutes and resuspend via sonication in appropriate buffer to maintain the pH of rNPs.
7. Optional: autoclave batch at 121° C. for 15 minutes.
8. Acquire CSR, ascertain rNP concentration, and perform functional QC adsorption assay.

### Example 1.6. Nanoclay Refinement Alternatives and Extensions

In addition to chemical conditioning or washing steps, refinement may optionally incorporate mechanical preprocessing operations designed to reduce particle size and liberate individual nanotubes from bulk aggregates. Such operations can include, but are not limited to, milling, sieving, and particle classification. These methods can be applied prior to, in conjunction with, or following chemical washes, and serve to (i) increase the accessible surface area, (ii) reduce the presence of quartz, feldspar, or other coarse contaminants, and (iii) provide a narrower particle size distribution that enhances the formation of colloidal suspensions. While not always required, these steps can significantly accelerate the liberation of nano-sized fractions and improve the consistency of subsequent chemical or dispersive treatments. The choice of mechanical preprocessing depends on the raw clay source, the intended downstream use of the refined particles, and the degree of purity or colloidal stability desired.

### Example 1.7. Continuous and Scaled Operations

Refinement steps may be implemented in continuous mode using inline mixers, static mixers, or high-shear devices, followed by continuous settlers, hydrocyclones, continuous centrifuges, or crossflow filtration for classification and concentration. Process parameters (flow, residence time, energy input) are selected to achieve target size distribution, dispersion stability, and functional performance.

### Example 1.7.1. Inline Mixing

Refinement slurries can be dispersed via inline rotor-stator mixers or static mixers, tuned for energy density, shear rate, and residence time.

### Example 1.7.2. Continuous Separation

Solids and colloids may be classified using hydrocyclones, tubular bowl centrifuges, or continuous settlers.

### Example 1.7.3. Crossflow Filtration

Useful for both washing and concentrating refined particles without creating hard, compacted pellets.

### Example 1.7.4. Parameter Control

Flow rates, residence times, and energy inputs can be varied to control colloidal stability, CSR (sedimentation ratio), and functional surface performance.

### Example 1.8. Storage and Stability of Nanoclays

Refined suspensions may be stored at about 2-25° C. For long-term storage, lyophilization or spray-drying with suitable excipients may be employed; reconstitution yields a working suspension meeting the same CSR and functional criteria.

### Example 1.9. Alternative Processing Techniques

Any of the steps described herein may be omitted, reordered, or substituted with equivalent operations without departing from the scope of the present disclosure. In certain embodiments, only dispersion, a single conditioning step, and a single classification step are employed to generate acceptable rNP performance. In other embodiments, surface modification (for example, silanation, polymer association) or magnetization steps are included to tailor handling or separation without altering the fundamental mechanism of preferential contaminant capture.

### Example 1.10. Colloidal Stability Ratio (CSR) and Mass Concentration

The colloidal stability and concentration of raw or refined nanoclays can be determined by multiple complementary methods. The following provide exemplary approaches, though other similar techniques may also be employed.

CSR Determination can occur through the following steps:
1. Prepare a fresh suspension of raw or refined nanoclays at the desired working concentration.
2. Zero the spectrophotometer at 250 nm and 700 nm using water or other diluent. Alternative wavelength pairs may be selected as appropriate for the instrument or sample.
3. Dilute an aliquot into a clear cuvette or microplate, usually to a final concentration of 0.1-2.0 mg/mL.
4. Measure optical densities (ODs).
5. Calculate the CSR as the ratio of OD₂₅₀/OD₇₀₀.
6. Optional: Record values over time (minutes to hours) to assess relative stability.

Gravimetric mass concentration can occur through the following steps:
1. Pipette a defined volume of suspension (e.g., 1 mL) into a pre-weighed pan or vessel.
2. Heat at approximately 100° C. until water is fully evaporated.
3. Cool to ambient temperature and reweigh.
4. Subtract the pan weight to determine dry mass.
5. Express concentration as mg/mL.

Optional alternative analysis approaches can occur through the following steps:
1. Turbidity Measurements. Optical density at 600 nm or other wavelengths may provide rapid, relative estimates of particle concentration.
2. Dynamic Light Scattering (DLS). Size distributions and particle counts can be derived, allowing indirect mass concentration calculation.
3. Inductively Coupled Plasma (ICP) Spectroscopy. Elemental analysis (e.g., Al, Si) provides precise quantification of nanoclay content.
4. Zeta Potential Analysis. Measurement of particle surface charge provides information on dispersion stability and can be correlated with sedimentation or aggregation tendencies.

### Example 1.11. Biological Samples

The starting biological material may be derived from prokaryotic or eukaryotic cells, including but not limited to: Escherichia coli or other bacterial hosts; yeast species such as Saccharomyces cerevisiae or Pichia pastoris; mammalian cells that harbor circular DNAs such as plasmid DNA, minicircle DNA, or eccDNA; and plant cells or algal cultures.

Samples may be obtained from laboratory-scale cultures, pilot-scale fermentations, or full-scale large volume manufacturing processes. Lysates can be prepared by alkaline lysis, detergent-mediated disruption, enzymatic digestion, mechanical disruption, or combinations thereof.

### Example 1.12. Cell Extract or Lysate Preparation

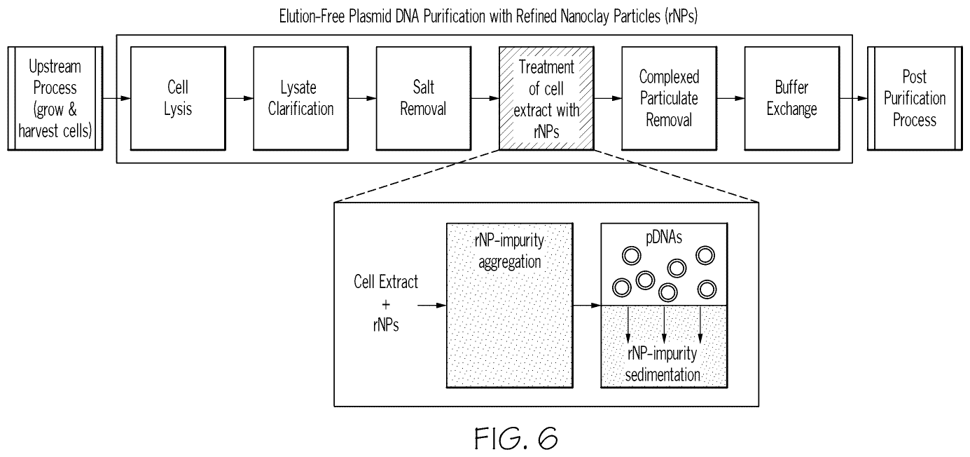

A workflow for use with extracts or lysates is shown in FIG. 6. Cell extracts or lysates suitable for use with the present invention may be generated by standard techniques, including alkaline SDS/NaOH lysis for plasmid preparations, detergent/organic extraction of total DNA, enzymatic wall digestion (e.g., EDTA-lysozyme for bacteria; lyticase/zymolyase for yeast), and mechanical disruption such as ultrasonication, bead milling, or high-pressure homogenization/French press. The appropriate method may be selected based on cell type, scale, and downstream requirements. The refined nanoclay particles described herein can be tuned to operate across these lysate modalities by adjusting pH, ionic strength, and dose to maintain preferential capture of non-plasmid components while leaving circular pDNA in solution.

A protocol for an alkaline lysis lysate preparation (illustrative, non-limiting) can include:
1. Grow Escherichia coli cells containing the target plasmid to mid- or late-log phase in LB medium supplemented with the appropriate antibiotic.
2. Harvest cells by centrifugation (e.g., 4,000×g, 10 min, 4° C.) and discard the supernatant.
3. Resuspend the cell pellet in isotonic buffer (e.g., 50 mM Tris-HCl, 10 mM EDTA, pH 8.0) at a volume of approximately 1/25- 1/50 the original culture volume.
4. Add alkaline lysis solution (e.g., 0.2 N NaOH, 1% SDS) at an equal volume to the resuspension buffer. Mix gently by inversion and incubate for approximately 3-5 minutes at room temperature to lyse cells and denature DNA and proteins.
5. Add chilled neutralization solution (e.g., 3 M potassium acetate, pH˜5.5) at an equal volume to precipitate genomic DNA, proteins, and cellular debris. Mix gently by inversion.
6. Clarify the lysate by centrifugation (e.g., 15,000×g, 10 min, 4° C.).
7. Collect the supernatant, which contains plasmid DNA along with residual RNA, genomic DNA, proteins, and other biomolecules.
8. Add 0.6 volumes of isopropyl alcohol to mixture to precipitate DNA.
9. Centrifuge at 15,000×g for 5 minutes to pellet DNA and remaining impurities.
10. Remove supernatant and wash pellet with 70% ice cold ethanol.
11. Dry pellet and resuspend in appropriate volume of chosen buffer (e.g., 10 mM Tris, pH 8.5)

### Example 1.13. Core Method for the Purification of pDNA from Cell Extracts

Conventional methods for plasmid DNA purification rely on chromatographic separations, such as ion-exchange, hydrophobic interaction, or size-exclusion, each of which requires multiple binding and washing steps followed by a distinct elution phase to recover the product. These workflows are labor-intensive, often require harsh chemical conditions, and can compromise yield or structural integrity of the plasmid.

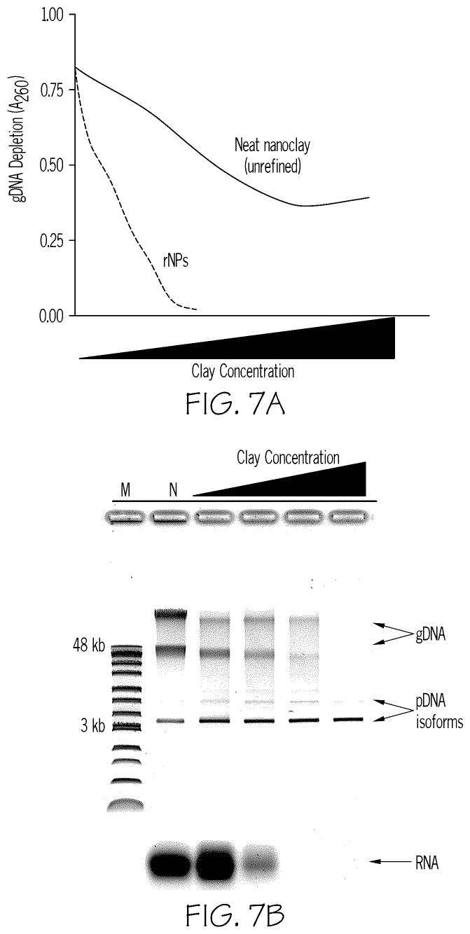

In contrast, the present method provides an elution-free purification strategy in which raw nanoclays or refined nanoclay particles (rNPs) are introduced directly into a plasmid-containing cell extract. Examples of output results from this elution-free workflow is illustrated in FIG. 7, where increasing amounts of rNPs leads to removal of undesirable genomic DNA (gDNA) and RNA. Tests using spectrophotometric assays with pure gDNA and tests using gel electrophoresis of mixtures containing gDNA, pDNA and RNA are shown in FIGS. 7A and 7B). Under controlled conditions, the particles selectively adsorb impurities—including genomic DNA, RNA, protein, and endotoxin—while leaving plasmid DNA substantially unbound in the supernatant. The loaded particles are then removed by physical separation, eliminating the need for gradient elution, column apparatus, or fraction collection. This fundamental departure from chromatographic paradigms enables a streamlined, scalable, and gentler purification of plasmid DNA. In one embodiment, the generic workflow includes the following:
1. Preparation of the nanoclay suspension:
   Nanoclay suspensions (raw or refined) are prepared by methods described elsewhere herein.
   These suspensions are then formulated to specific pH, conductivity, and mass concentration before being applied to cell extracts under conditions that favor impurity adsorption while leaving plasmid DNA substantially in solution.
   Establishment of nanoclay adsorption strength using dose determination module.
2. Contacting the pDNA harboring sample:
   Mix the nanoclay suspension with a clarified or unclarified cell lysate or extract. In some embodiments, nanoclay is added to the sample while in others the sample is added to the nanoclay suspension.
   Typical mass ratios of nanoclay to total nucleic acid or biomolecular components may be expressed in several ways depending on the context. For example, in weight-to-weight (w/w) terms, ratios may range from about 1:1 to over 100,000:1, with higher ratios often employed to ensure complete adsorption of highly concentrated non-target biomolecules. In concentration terms, nanoclay suspensions may be used at levels ranging from as low as approximately 0.1 mg/mL to over 100 mg/mL. In absorbance-normalized terms, amounts on the order of 5.0×10⁻⁵ g of nanoclay per A₂₆₀ unit of nucleic acid can be employed, although higher or lower values may be used. Selection of concentration or ratio will depend on the sample type, target selectivity, process configuration, and scale of operation.
   Incubate for a period ranging from seconds to several hours, with mixing or agitation to ensure uniform contact.
3. Separation of nanoclay-contaminant complexes:
   Remove the clay particles containing bound contaminants by centrifugation, sedimentation, filtration, or magnetic capture (for magnetically modified nanoclays).
   The supernatant or filtrate contains the target pDNA.
4. Optional downstream steps:
   Treatment of the clarified pDNA-containing solution with an RNase under conditions that selectively degrade residual RNA without damaging pDNA. If not permitted due to purity restrictions, this step may be omitted.
   Recovery and concentration of pDNA using precipitation methods (e.g., isopropanol or ethanol precipitation with or without salt carriers) or by membrane-based techniques such as ultrafiltration/diafiltration. Choice of method may depend on downstream application, required purity, and desired yield.
   Adjustment of buffer composition and ionic strength, optionally combined with pH optimization, to meet target storage or application requirements. This can include buffer exchange into physiological or low-salt buffers, removal of low-molecular-weight impurities, and passage through sterile filtration devices (e.g., 0.22 μm) to achieve sterility for clinical or GMP-grade use.

### Example 1.14. Dose Determination as a Modular Step

The effective amount of nanoclay required to purify plasmid DNA (pDNA) from cell extracts cannot be fixed as a single universal value, because the adsorption process is influenced by multiple variables intrinsic to both the biological material and the nanoclay itself. Factors such as plasmid copy number per cell, overall nucleic acid concentration, extract conductivity or ionic strength, the specific lysis protocol employed (e.g., alkaline, enzymatic, detergent, or mechanical disruption), and the lot-to-lot adsorption activity of the nanoclay all play significant roles in determining the final dose.

For this reason, dose determination is treated as a modular component of the purification workflow. Prior to scaling a batch, each batch of raw nanoclay or rNPs is first standardized against a small aliquot of representative material. In some embodiments, this material is a true cell extract reflecting the process of interest. In other embodiments, the material is a synthetic pool containing known amounts of genomic DNA, RNA, lipopolysaccharide (LPS), and proteins. The test system provides a controlled matrix in which adsorption behavior can be assessed rapidly and reproducibly.

During this qualification assay, a series of doses of nanoclay are added to the test sample under the intended buffer and mixing conditions. After short incubations, the clarified supernatant is analyzed by UV absorbance (A_{260/280} ratio, turbidity at A₃₂₀), electrophoresis, or specific assays for RNA, protein, or endotoxin. A dose-response curve may be generated, when desired, to establish the relationship between the mass of nanoclay applied and the extent of contaminant removal.

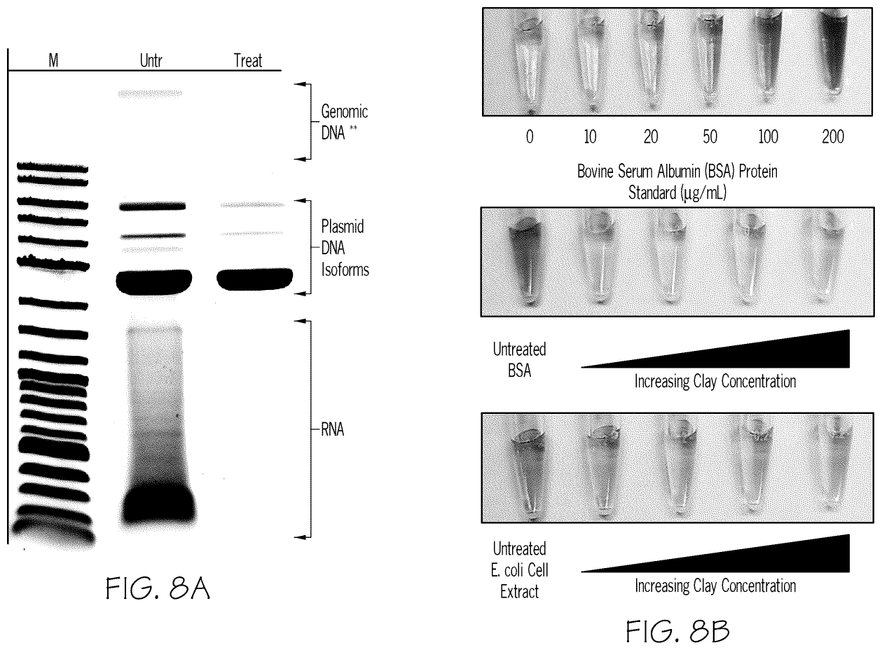

In some embodiments, this is expressed in terms of the mass of nanoclay required to adsorb a defined mass of a particular biomolecule, such as genomic DNA, RNA, protein, or lipopolysaccharide. In other embodiments, a global optical measure such as absorbance at 260 nm (A₂₆₀) is employed as a proxy for the total nucleic acid or biomolecular burden of the extract. Plotting nanoclay dose against the reduction in contaminant signal (e.g., decrease in A₂₆₀, reduction in RNA band on a gel, or decrease in the purple color produced in the bicinchoninic acid (BCA) protein assay) yields a characteristic dose-response profile. A representative dose-response analysis is shown in FIG. 8. Removal of nucleic acids from an extract is shown in FIG. 8A. In FIG. 8B agarose gel electrophoresis demonstrates rNP concentration-dependent removal of proteins from a cell extract.

By defining dose determination as a stand-alone, repeatable module, the purification method remains adaptable across diverse process conditions. Whether the extract arises from high-copy plasmid E. coli, low-copy constructs, yeast, or mammalian cells; whether the ionic background is low-salt or high-salt; and whether the nanoclay is raw, refined, or functionally modified, the same modular assay ensures that the appropriate clay input is empirically determined before committing to production scale. This approach accommodates inherent variability while ensuring consistent plasmid yield, purity, and reproducibility across lots and applications.

### Example 1.15. Nanoclay Dose Screen (100 μL or 1 mL Format)

This Example aimed to find the minimum nanoclay concentration that removes ≥90% of total nucleic-acid signal (by A₂₆₀ and/or AGE) of a chosen standard (e.g., genomic DNA, plasmid preparation). The minimum nanoclay concentration can then be converted to a dose factor to be used for production samples.

The test standards included purified genomic DNA or plasmid DNA, or protein standards. In alternative embodiments, a representative plasmid DNA preparation containing typical impurities (e.g., RNA, protein, genomic DNA, and/or endotoxin) may be used as the test substrate.

The buffer matching end use may include 10-50 mM Tris, pH 7.5-9.0 and salt per use case. The nanoclay stock (raw or rNPs) may include 10-100 mg/mL in a chosen buffer (vortex/sonicate to disperse). A UV-Vis Spectrophotometer or fluorometer may be used (e.g., Qubit).

Various agarose gel electrophoresis systems may be used. Alternatively, capillary gel electrophoresis (CGE) or high-performance liquid chromatography (HPLC) can be used to detect and quantify biomolecules.

### Example 1.15.1. Procedure (Example, E. coli gDNA Standard)

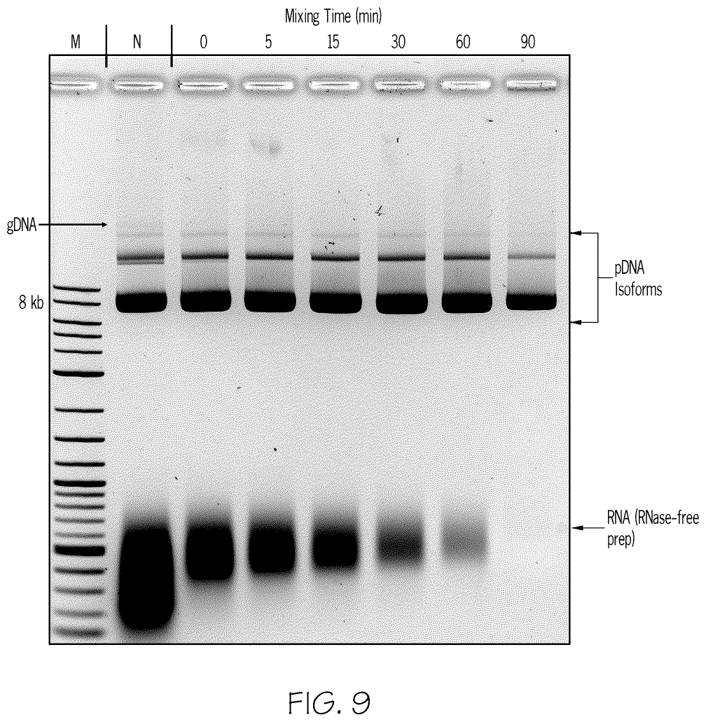

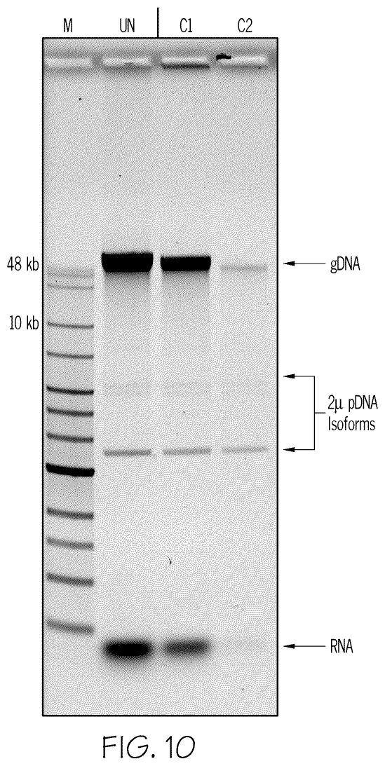

A volume format may be selected, such as a 100 μL (low-waste) or 1 mL format. Example test concentrations (e.g., final, in the reaction) can include 0 (control), 1, 2, 5, 10, or 20 mg/mL final nanoclay concentration. The tests can be done in duplicate or triplicate, if necessary. The tests may conduct reactions at room temperature (18-25° C.), such as under the following protocol:
1. Transfer a sufficient volume of gDNA stock into a fresh 1.5 mL microcentrifuge tube such that, after dilution, the final concentration will be approximately 50 ng/μL (A₂₆₀˜1.0).
2. Dilute the DNA with the appropriate buffer (e.g., 10 mM Tris, pH 8.0, or another buffer matching the intended use case) according to the test nanoclay concentrations.
3. Mix gently by flicking or brief vortexing, then spin down briefly to collect the solution at the bottom of the tube.
4. Transfer the appropriate volume of test nanoclay to reach the appropriate final concentration for each sample and vortex for 5-10 seconds to mix. Longer times and different mixing strategies may be employed as necessary. An example of the usefulness of optimizing mixing times is shown in FIG. 9.
5. Remove clay-contaminant complexes by centrifugation at 20,000×g for 10 minutes.
6. Carefully transfer supernatants to fresh tubes.
7. Obtain optical density data at 260 nm, 280 nm, and 320 nm for the supernatants and compare it to a clay-free control to determine percentage removal. The OD₃₂₀ value is required to ensure particulates are properly removed from the supernatants. If ≥0.03, centrifuge for longer or consider filtering through a 0.1-0.22 μm membrane filter before remeasuring.
8. Optional: Use rNPs to purify circular DNAs from cells of more complex eukaryotes such as the yeast Saccharomyces cerevisiae and other haploid or diploid cells. An example of such a purification is shown in FIG. 10, where a circular DNA called the 2μ plasmid was extracted from yeast cells.

### Example 1.15.2. End-Point PCR for Detection of Residual Genomic DNA (Nanoclay Functional Test)

This Example demonstrates preferential removal of genomic DNA (gDNA) by rNPs or raw powder suspensions in a simple test matrix and confirms removal by endpoint PCR of a host-locus amplicon. The materials include: E. coli or other host gDNA or a pDNA-harboring cell extract or lysate; nanoclay stock suspension; DNase/RNase-free water; a microcentrifuge capable of 20,000× g; spectrophotometer and cuvettes (or microvolume device); agarose gel electrophoresis setup and stain; endpoint PCR reagents: polymerase master mix, DNA primers specific to a single-copy host locus; and a thermocycler.

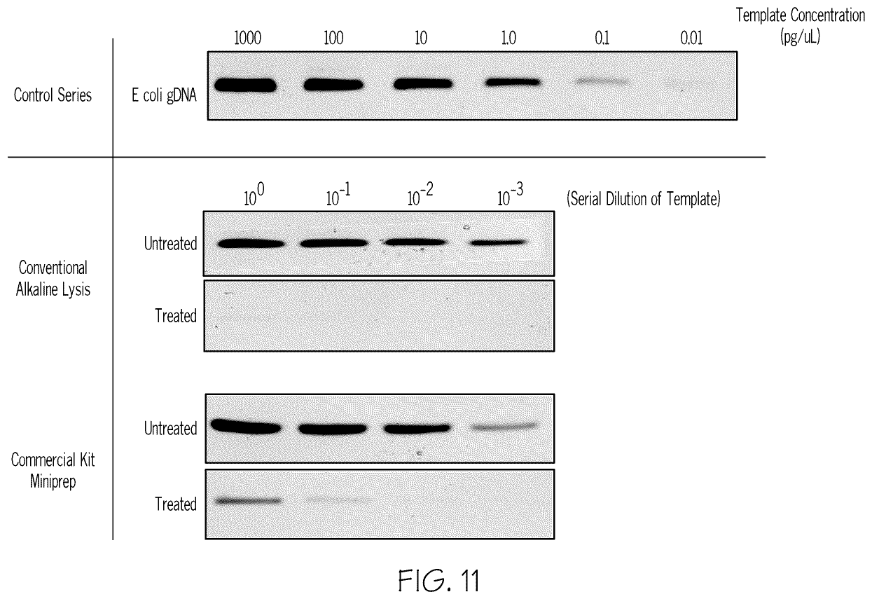

Endpoint PCR may be used to verify reduction of host genomic DNA from cell extracts. Reactions are assembled with a polymerase master mix and primers targeting a host genomic locus. Template materials may be drawn from any suitable source, including but not limited to rNP-treated supernatants, untreated control supernatants, purified or crude host gDNA preparations, and defined synthetic lysate standards. Templates may be used neat, diluted, or concentrated, and equivalent fractions obtained by alternative separation steps (e.g., depth filtration, and/or low-g clarification) may be substituted as desired. A typical result from gel electrophoresis analysis to detect residual gDNA is shown in FIG. 11.

Typical reactions (10-50 μL) are cycled for 25-35 cycles using standard conditions appropriate to the primer Tₘ. Amplicons are resolved by agarose gel electrophoresis and visualized with a DNA stain. Interpretation is based on presence, absence or relative intensity of the expected band. Dilution of templates may be employed to mitigate potential PCR inhibitors.

Appropriate controls (no-template, positive control DNA, and a matrix-only control processed without rNPs) may be included as desired. Below is a typical workflow for an end-point PCR assay.
1. Prepare primers (e.g., 10 μM forward and reverse) specific to a single-copy genomic locus.
2. Prepare templates and dilutions. For each condition (treated and control), generate either:
   a) a serial 10-fold dilution series of the supernatant (e.g., undiluted, 1:10, 1:100, 1:1000), or
   b) a single multifold dilution from the base (e.g., 1:10 or 1:100), as desired. Use nuclease-free water or PCR buffer for diluent; mix gently at each step.
3. Assemble duplicate PCRs for each dilution and condition with equal input volume per reaction (e.g., 1-2 μL template in a 25 μL reaction).
4. Include controls: no-template control (NTC) and a clay-only control (nanoclay-treated water or buffer processed through the same steps as test samples).
5. Run endpoint PCR under standard conditions (e.g., 25-35 cycles; annealing temperature chosen per primer Tm; polymerase instructions).
6. Resolve amplicons on an agarose gel alongside a DNA ladder; obtain gel image using e.g., ethidium bromide staining under identical the exposure settings.
7. Interpret results. Expect a band in the untreated control across more dilutions than in the treated sample; loss or marked reduction of the band in the treated series at equal dilution indicates successful gDNA removal. Record the highest dilution showing a clear band for each condition.
8. If inhibition is suspected (e.g., weak or absent bands across all lanes), increase dilution (e.g., 1:5 to 1:10 or extend the 10-fold series), or clarify templates by a brief spin at 20,000×g for 5 minutes before rerunning.

### Example 1.15.3. Decision Rule

In this example procedure, the lowest dose giving ≥90% removal is the effective dose for that lot in that buffer/salt/mixing mode. One may convert dose screen data to a reusable dose factor (DF). The dose screen provides valuable insight on the adsorption strength of a particular nanoclay batch and the underlying reaction conditions. However, since cell extracts and lysates contain multiple biomolecular species that absorb at 260 nm, it isn't all that useful in estimating impurity levels and remaining pDNA after purification. For this reason, it can be useful to convert the mass of gDNA into the total absorbance units (a.u.) in each reaction.

By calculating the amount of nanoclay required to achieve a defined reduction in A₂₆₀ (for example, ≥90% removal relative to a clay-free control), a dose factor (DF) can be established, expressed as grams (or mg) of nanoclay per A₂₆₀ unit of sample. This dose factor provides a generic, load-based metric that can be scaled to different extracts, buffer conditions, or process volumes, allowing each new batch of nanoclay to be standardized for effective use regardless of variation in sample composition.

In addition, the purity of plasmid DNA in cell extracts can be conveniently monitored by the ratio of absorbance at 260 nm to 280 nm (A260/280), which provides a simple optical indicator of nucleic acid versus protein content. Because RNA is often the predominant contributor to impurity burden and strongly influences the A260 signal, monitoring the total absorbance load in combination with the A260/280 ratio makes the dose determination process even more prescient.

Example Calculation—90% removal of gDNA with 5 mg/mL nanoclay
Initial gDNA concentration: 50 ng/μL
Volume of test reaction: 100 μL
Total gDNA mass in reaction:

$$50 ng / µL \times 100 µL = 5 , 000 ng$$

$$Total {A}_{260} = 5000 ng \times 1 a . u . / 50 ng = 100 a . u .$$

### Dose Applied:

Effective nanoclay concentration: 5 mg/mL
Reaction volume: 100 μL.
Mass of nanoclay in reaction:

$$5 mg / mL \times 0.1 mL = 0.5 mg = 5. \times {10}^{- 4} g nanoclay$$

### Dose Factor Calculation:

Total A₂₆₀ units (control): 100 a.u.
Clay mass used: 0.5 mg=5.0×10⁻⁴ g

$${D}_{F} = nanoclay mass / total a . u . = 5. \times 1 {0}^{- 4} g / 100 a . u . = 5. \times {10}^{- 6} g / a . u .$$

Example pDNA Purification Starting Dose Calculation (Illustrative)

$$Cell Extract = 100 mL ( 100 a . u . / µL )$$

$$Nanoclay {D}_{F} = 5. \times {10}^{- 6} g / a . u .$$

$$Total a . u . = 100 , 000 µL \times 100 a . u . / µL = 10 , 000 , 000 a . u .$$

$$Total nanoclay to use = 10 , 000 , 000 a . u . \times 5. \times {10}^{- 6} g / a . u . = 50 g$$

### Example A—Purification of pDNA from a Low Conductivity Cell Extract (˜0.1-1.0 mS/cm)

$$Cell Extract = 100 mL ( 100 a . u . / µL )$$

$$Nanoclay {D}_{F} = 5. \times {10}^{- 6} g / a . u .$$

$$Total nanoclay to use = 10 , 000 , 000 a . u . \times 5. \times {10}^{- 6} g / a . u . = 50 g$$

1. Add 50 g of rNPs to the 100 mL cell extract buffered in 10 mM Tris (pH 8.0).
2. Place on a rotating mixer at 20 rpm.
3. Incubate the reaction for ˜10 minutes at 18-25° C. and then collect an aliquot of the mixture (e.g., 200 μL). Continue to mix.
4. Pellet nanoclay-contaminant complexes from the aliquoted sample at ˜20 k×g for 10 minutes.
5. Assay the supernatant at 260 nm, 280 nm, and 320 nm as described above. Record the values and calculate the A_{260/280} purity. If ≥1.95, add 25% of the original mass of rNPs and continue mixing for 30 minutes.
6. Collect another aliquot and repeat steps 4-5 until ratio drops to near 1.90.
7. When the ratio drops to near 1.90, add 10% more nanoclay and rotate mix for 30 minutes. If 1.90 is sufficient for the process, skip this and move to step 9.
8. Collect another aliquot and repeat steps 4-5 again. Once around 1.85, it is advised to allow the mixture to continue mixing until target purity is achieved.
9. Pellet the remaining clay-bound impurities by centrifugation at 20,000×g for 15 minutes.
10. Carefully extract the supernatant containing plasmid DNA and transfer it to a fresh container.
11. Concentrate or buffer-exchange as needed for downstream use and analyze final batch according to required QCs.

### Example B—Purification of pDNA from a High Conductivity Cell Extract (>10 mS/cm)

$$Cell Extract = 100 mL ( 100 a . u . / µL )$$

$$Nanoclay {D}_{F} = 1. \times {10}^{- 6} g / a . u .$$

$$Total nanoclay to use = 10 , 000 , 000 a . u . \times 1 \text{.0} \times {10}^{- 6} g / a . u . = 10 g$$

1. Add 10 g of rNPs (pH 8.0) to the 100 mL of cell extract buffered in 100 mM Tris (pH
8.0).
2. Place on a rotating mixer at 20 rpm.
3. Incubate the reaction for ˜10 minutes at 18-25° C. and then collect an aliquot of the mixture (e.g., 200 μL). Continue to mix.
4. Pellet nanoclay-contaminant complexes from the aliquoted sample at ˜20 k×g for 10 minutes.
5. Assay the supernatant at 260 nm, 280 nm, and 320 nm as described above. Record the values and calculate the A_{260/280} ratio. If ≥1.95, add 25% of the original mass of rNPs and continue mixing for 30 minutes. If near 1.90, move to step 7.
6. Collect another aliquot and repeat steps 4-5 until purity drops below 1.90.
7. When the purity drops to near 1.90, add 10% more nanoclay and rotate mix for 30 minutes. If 1.90 is sufficient for the process, move to step 9.
8. Collect another aliquot and repeat steps 4-5 again. Once around 1.85, it is advised to allow the mixture to continue mixing until target purity is achieved.
9. Pellet the remaining clay-bound impurities by centrifugation at 20,000×g for 15 minutes.
10. Carefully extract the supernatant containing plasmid DNA and transfer it to a fresh container.
11. Concentrate or buffer-exchange as needed for downstream use and analyze final batch according to required QCs.

### Example 1.16. Process Modes

The methods of the present disclosure can be implemented in:
Batch mode: all components combined in a single vessel.
Staged mode: nanoclay treatment before or after another purification step.
Continuous mode: inline mixing of lysate with nanoclay slurry followed by continuous-flow separation in a closed, single-use system.

### Example 1.17. Kits and Ready-to-Use Formats

In certain embodiments, the invention provides pDNA purification kits comprising:
Pre-measured or declared doses of nanoclay (refined or unrefined) in dry or suspension form.
Buffer concentrates for optimal pH and ionic strength adjustment.
Instructions for use in bacterial, yeast, or mammalian systems.
Optional accessories for separation (e.g., syringe filters, magnetic racks).

### Example 1.18. Discussion

Nanoparticles are materials having at least one dimension of approximately 100 nanometers or less, conferring structural and functional properties that differ markedly from those of their bulkier counterparts. At this scale, particles exhibit exceptionally high surface-area-to-mass (or surface-area-to-volume) ratios, dramatically enhancing their chemical reactivity and capacity for molecular interactions. Such features enable nanoparticles to engage with biological macromolecules—including nucleic acids, proteins, and polysaccharides—in ways not observed for larger particles, creating unique opportunities for separation and binding phenomena.

A broad class of nanoparticles includes aluminosilicate minerals, which encompass a diverse family of naturally occurring and synthetic materials composed primarily of aluminum, silicon, oxygen, and, in some cases, hydroxyl groups and water. Within this family are layered and framework phyllosilicates, tubular aluminosilicates, hydrated aluminum silicates, and fibrous silicate minerals. Representative examples include kaolinite group minerals (e.g., kaolin, halloysite, dickite, nacrite), smectites (e.g., montmorillonite, beidellite, hectorite, saponite), sepiolite, palygorskite, attapulgite, and synthetic analogs such as laponite. These materials present morphologies ranging from plate-like to fibrous to tubular, with internal and external surfaces differing in composition, polarity, and charge distribution.

One member of the kaolinite group, halloysite nanotubes (HNTs), illustrates the distinctive structural and surface chemistry possible within aluminosilicates. Halloysite is a naturally occurring aluminosilicate mineral with the chemical formula Al₂Si₂O₅(OH)₄·nH₂O. The mineral forms hollow tubular particles with typical lumen diameters of about 15 nm, external diameters ranging from 50-70 nm, and lengths spanning hundreds of nanometers to several microns. The multilayered walls consist of alternating alumina and silica layers: the lumen-facing surface is lined with hydroxyl groups bound to alumina, while the outer surface consists of siloxane (Si—O—Si) linkages. Because of the differing dielectric and ionization properties of these oxide surfaces, the lumen and outer surfaces carry opposite charges in aqueous environments at pH values between 3 and 8, enabling selective electrostatic and hydrogen-bond interactions with a variety of biomolecules.

Although nanoclays have been investigated for the adsorption of individual classes of biomolecules—for example, binding DNA in the presence of salts, removing proteins from solution, or interacting with RNA under defined conditions—these studies have largely been conducted in simplified systems containing a single biomolecule type. To the knowledge of the Applicant, no prior work has examined or optimized nanoclay behavior in complex biological mixtures containing multiple biomolecule classes simultaneously, such as those present in crude cell lysates or extracts, for the purpose of isolating or purifying a particular target biomolecule. The discovery that certain nanoclays can exhibit pronounced selectivity for non-circular nucleic acids and other contaminants, while leaving supercoiled plasmid DNA and related circular DNAs unbound under competitive conditions, was unexpected. Moreover, the refinement of raw nanoclay powders into reproducible, colloidally stable nanoparticles (rNPs) proved to be critical in achieving this selectivity. The ability of rNPs to maintain their dispersion, avoid aggregation, and present a consistent high-surface-area interface enabled an entirely new separation behavior not predictable from raw powder suspensions or from the current body of literature. Importantly, this refinement-driven reproducibility was itself innovative, as it unlocked selectivity that could not be anticipated from unrefined clays. This combination of refinement-driven reproducibility and selective adsorption in biologically complex environments represents an innovative and unforeseen advance over prior art.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of purifying circular DNA from a sample, said method comprising:
exposing the sample to a nanoclay, wherein the nanoclay preferentially binds to one or more non-circular DNA components of the sample relative to the circular DNAs in the sample; and
separating the exposed sample from the nanoclay, wherein the separated sample comprises purified circular DNA.

2. The method of claim 1, wherein the sample comprises a cellular extract selected from the group consisting of a bacterial cell extract, a yeast cell extract, a eukaryotic cell extract, a mammalian cell extract, or combinations thereof.

3. The method of claim 1, wherein the exposing comprises mixing the sample and the nanoclay under conditions sufficient to promote contact between the nanoclay and the non-circular DNA components in the sample.

4. The method of claim 1, wherein the separating occurs by a method selected from the group consisting of centrifugation, filtration, gravity sedimentation, magnetic separation, or combinations thereof.

5. The method of claim 1, wherein the nanoclay is functionalized with magnetic materials to render them magnetically responsive, and wherein the separating comprises magnetic separation through magnetic capture of the nanoclay.

6. The method of claim 1, wherein the nanoclay is selected from the group consisting of halloysite, montmorillonite, sepiolite, laponite, kaolin, kaolinite, silicates, aluminosilicates, metakaolin, dickite, nacrite, smectite clays, bentonite, beidellite, nontronite, hectorite, saponite, palygorskite, attapulgite, phyllosilicates, or combinations thereof.

7. The method of claim 1, wherein the nanoclay comprises refined nanoclay particles (rNPs), wherein the rNPs are refined by washing nanoclays with a solution selected from the group consisting of basic solutions, acidic solutions, chelating agents, detergents, organic solvents, or combinations thereof.

8. The method of claim 1, wherein the amount of nanoclay is determined by a dose factor (DF) expressed as grams of nanoclay per A₂₆₀ unit of nucleic acid in the sample.

9. The method of claim 8, wherein the DF is established through a dose-response screen using a reference sample.

10. The method of claim 1, wherein the purified circular DNA exhibits an A₂₆₀/A₂₈₀ ratio of at least 1.80-1.90.

11. The method of claim 1, wherein the nanoclay comprises aluminosilicate materials.

12. The method of claim 1, wherein the circular DNA is selected from the group consisting of extrachromosomal circular DNAs (eccDNAs), plasmid DNA (pDNA), viral episomes, synthetic minicircle DNAs, or combinations thereof.

13. The method of claim 1, wherein the circular DNA comprises plasmid DNA (pDNA).

14. The method of claim 1, wherein the circular DNA remains substantially unbound in the sample.

15. The method of claim 1, wherein the non-circular DNA components are selected from the group consisting of genomic DNA, chromosomal DNA, linear DNA fragments, degraded nucleic acids, RNA, messenger RNA, ribosomal RNA, transfer RNA, small RNAs, proteins, host cell proteins, endotoxins, polysaccharides, lipopolysaccharide (LPS), carbohydrates, lipids, metabolites, cellular debris, contaminating components, impurities, or combinations thereof.

16. The method of claim 1, wherein the method is repeated multiple times, and wherein the method comprises:
(a) exposing the sample to the nanoclay;
(b) separating the exposed sample from the nanoclay;
(c) re-exposing the separated sample to the nanoclay; and
(d) separating the re-exposed sample from the nanoclay, wherein the first exposing step occurs under higher salt concentrations than the second re-exposing step.

17. The method of claim 1, wherein the method occurs in an elution-free manner.

18. A system operable for purifying circular DNA from a sample, wherein the system comprises:
a composition comprising a nanoclay, wherein the nanoclay is operable to preferentially bind to one or more non-circular DNA components of the sample relative to the circular DNAs in the sample; and
a vessel containing the nanoclay.

19. The system of claim 18, wherein the vessel comprises an agitator operable to mix the sample with the nanoclay.

20. The system of claim 18, wherein the nanoclay is in the form of a dispersed suspension in an aqueous buffer in the vessel.

21. The system of claim 18, wherein the nanoclay is in the form of a dry powder operable to be reconstituted in a buffer.

22. The system of claim 18, wherein the system is in the form of a kit, wherein the kit further comprises instructions for using the nanoclay to purify circular DNA from the sample.

23. The method of claim 18, wherein the nanoclay is selected from the group consisting of halloysite, montmorillonite, sepiolite, laponite, kaolin, kaolinite, silicates, aluminosilicates, metakaolin, dickite, nacrite, smectite clays, bentonite, beidellite, nontronite, hectorite, saponite, palygorskite, attapulgite, phyllosilicates, or combinations thereof.

24. The method of claim 18, wherein the nanoclay comprises refined nanoclay particles (rNPs), wherein the rNPs are refined by washing nanoclays with a solution selected from the group consisting of basic solutions, acidic solutions, chelating agents, detergents, organic solvents, or combinations thereof.

25. The method of claim 18, wherein the nanoclay comprises halloysite nanotubes.

26. A method of refining a nanoclay for use in purifying circular DNA from a sample, said method comprising:
(a) washing raw nanoclay with a solution selected from the group consisting of basic solutions, acidic solutions, chelating agents, detergents, organic solvents, or combinations thereof; and
(b) dispersing the washed nanoclay; and
(c) isolating the nanoclay.

27. The method of claim 26, further comprising a step of
(d) characterizing the refined nanoclay.

28. The method of claim 26, wherein the nanoclay is washed with a basic solution, wherein the basic solution is selected from the group consisting of NaOH, KOH, NH₄OH, or combinations thereof.

29. The method of claim 26, wherein the isolating comprises controlled sedimentation or ultrafiltration to select a desired size range.

30. The method of claim 26, wherein the nanoclay is selected from the group consisting of halloysite, montmorillonite, sepiolite, laponite, kaolin, kaolinite, silicates, aluminosilicates, metakaolin, dickite, nacrite, smectite clays, bentonite, beidellite, nontronite, hectorite, saponite, palygorskite, attapulgite, phyllosilicates, or combinations thereof.

31. The method of claim 26, wherein the nanoclay is functionalized with cationic polymers, peptides, or ligands to enhance adsorption of non-circular DNA components.

32. The method of claim 27, wherein the nanoclay is characterized by a colloidal scattering ratio (CSR) at least two-fold higher than the corresponding raw nanoclay.
